(19) United States
(12) Patent Application Publication
(10) Pub. No.: US 2010/0105071 A1
(43) Pub. Date: Apr. 29, 2010

(54) METHODS FOR PREDICTING THE ONSET OF MENARCHE

(75) Inventors: Marc Laufer, Weston, MA (US); Henry Feldman, Newton, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/528,814

(22) PCT Filed: Feb. 27, 2008
(86) PCT No.: PCT/US08/02566

§ 371 Date: Nov. 16, 2009

Related U.S. Application Data
(60) Provisional application No. 60/904,039, filed on Feb. 28, 2007.

(51) Int. Cl. G01N 33/53; G01N 33/543

(52) U.S. Cl. 435/7.1; 436/518

(57) ABSTRACT

Embodiments of the invention provide methods for predicting the onset of menarche, i.e. a females first menstrual period, through the measurement of salivary hormone levels (e.g. 17-β estradiol, testosterone, progesterone, and/or 17 hydroxy-progesterone (17-OHP)). In particular, the methods described herein enable a reliable determination that menarche will, or will not occur, within varied time intervals, e.g. 1 to 30 days, 1 to 45 days, 1 to 60 days and 1 to 90 days. Diagnostic kits and products of manufacture comprising the kits are also provided.

### CROSS REFERENCE TO RELATED APPLICATIONS

This application claims the benefit under 35 U.S.C. §119(e) of U.S. Provisional Application No. 60/904,039 filed Feb. 28, 2007.

### FIELD OF THE INVENTION

Embodiments of the invention described herein relate to methods for predicting the onset of menarche through the measurement of salivary hormone levels (e.g. 17-β estradiol, testosterone, progesterone, and/or 17 hydroxy-progesterone (17-OHP)). In particular, the methods described herein enable a reliable determination that menarche will, or will not occur, within an identified time interval, e.g. 1 to 30 days, 1 to 45 days, 1 to 60 days and 1 to 90 days. Diagnostic kits and products of manufacture comprising these kits are also provided.

### BACKGROUND OF THE INVENTION

Many girls and young women would like to be able to predict the onset of menses. Currently all that clinicians can predict is the usual onset of menses within two years of the onset of breast development.

Prediction of menarche is commonly based on clinical conjecture, using an imprecise combination of Tanner staging, weight, and body-mass index (BMI). Although ovulation can be predicted in postpubertal women through changes in circulating ovarian hormone concentration most menstrual periods in the first two years after menarche are not preceded by ovulation. A clearer understanding of the biochemical events that induce anovulatory cycles, leading to more precise prediction of menarche, could be useful in clinical situations, ranging from evaluation of delayed puberty to decisions about inducing amenorrhea in pre-pubertal girls undergoing bone marrow transplant, and to anxiety over normal development. Thus, the ability to predict the onset of menses is important both from a medical standpoint e.g, to avoid the occurrence of bleeding during throbocytopenia of cancer chemotherapy; and from a psycho-social standpoint e.g., to decrease anxiety over timing of the occurrence of menarche such as when attending summer camp, or during a sports competition. There has been a long standing need to develop a rapid test procedure that will accurately predict the onset of menses within a short period of time, e.g. within two months.

### SUMMARY OF THE INVENTION

The present invention provides methods for predicting the onset of menarche in girls and young women within short time intervals including, but not limited to, 1 to 30 days, 1 to 45 days, 1 to 60 days and 1 to 90 days. The diagnostic methods involve measuring the level of at least one hormone (e.g. 17-β estradiol, testosterone, progesterone, and/or 17 hydroxy-progesterone (17-OHP)) in saliva from a female. In one aspect of the invention, the measured hormone levels are compared to a predetermined threshold level, H*, for at least one hormone, where a measured hormone level above the threshold hormone level indicates that menarche will occur within the specified interval. A measured hormone level below a predetermined threshold hormone level indicates that menarche will not occur in the specified time interval. In another aspect of the invention, the measured level is applied to a multivariate prediction formula specifically constructed for an indicated time interval in order to obtain a prediction value (L), where a L value less than a threshold L* value indicates that menarche will not occur within the time interval and a L greater than the threshold L* value indicates that menarche will occur within the specified time interval.

In one embodiment, a method of predicting that menarche is not anticipated within a known time interval in a human female is provided. The method comprises a) measuring the level of at least one hormone in a saliva sample from said female; and b) comparing the level of at least one hormone to a selected H* value to determine if menarche is not anticipated within said known time interval.

In one embodiment, the hormone to be measured in step a) is selected from the group consisting of testosterone, 17-beta estradiol, progesterone, 17-α hydroxyprogesterone, and combinations thereof.

In one embodiment, the female is in Tanner Stage III-IV.

In one embodiment, the hormone to be measured is testosterone and the selected H* value for testosterone is in the range between 0.01 and 0.05 nanograms per milliliter (ng/ml). If the measured value of testosterone does not exceed said selected H* value for testosterone then menarche is not anticipated within said known time interval.

In one embodiment, the hormone to be measured is 17-beta estradiol and the selected H* value for 17-beta estradiol is in the range between 0.5 and 10.0 picograms per milliliter (pg/ml). If the measured value of 17-beta estradiol does not exceed said selected H* value for 17-beta estradiol then menarche is not anticipated within said known time interval.

In one embodiment, the hormone to be measured is progesterone and the selected H* value for progesterone is in the range between 0.01 and 0.05 ng/ml. If the measured value of progesterone does not exceed said selected H* value for progesterone then menarche is not anticipated within said known time interval.

In one embodiment, the hormone is 17-hydroxyprogesterone and the selected H* value for 17-hydroxyprogesterone is in the range between 0.01 and 0.10 ng/ml. If the measured value of 17-hydroxyprogesterone does not exceed said selected H* value for 17-hydroxyprogesterone then menarche is not anticipated within said known time interval.

In one embodiment, the saliva sample is collected by the female. In one embodiment, the female measures the level of at least one hormone in a saliva sample and compares the level of at least one hormone to a selected H* value to determine if menarche is not anticipated within said known time interval in her own home.

In one embodiment the steps of a) measuring the level of at least one hormone in a saliva sample from said female; and b) comparing the level of at least one hormone to a selected H* value to determine if menarche is not anticipated within said known time interval are completed with the use of an immunoassay. In one embodiment a visual color change of the immunoassay indicates a positive or negative result.

In one embodiment, the known time interval is between 1 to 60 days, or between 1 to 90 days, or between 1 to 45 days, or between 1 to 30 days.

In one embodiment, the saliva sample is obtained by spitting saliva into a tube, or by swabbing saliva from the mouth, or by depositing saliva onto a test strip.

In another embodiment, a method of predicting that menarche is anticipated within a known time interval in a human female is provided. The method comprises a) measuring the level of at least one hormone in a saliva sample from said female; and b) comparing the level of at least one hormone to a selected H* value to determine if menarche is anticipated within said known time interval.

In one embodiment, the hormone to be measured in step a) is selected from the group consisting of testosterone, 17-beta estradiol, progesterone, 17-α hydroxyprogesterone, and combinations thereof.

In one embodiment, the female is in Tanner Stage III-IV.

In one embodiment, the hormone to be measured is testosterone and the selected H* value for testosterone is in the range between 0.01 and 0.05 ng/ml. If the measured value of testosterone exceeds said selected H* value for testosterone then menarche is anticipated within said known time interval.

In one embodiment, the hormone to be measured is 17-beta estradiol and the selected H* value for 17-beta estradiol is in the range between 0.5 and 10.0 pg/ml. If the measured value of 17-beta estradiol exceeds said selected H* value for 17-beta estradiol then menarche is anticipated within said known time interval.

In one embodiment, the hormone to be measured is progesterone and the selected H* value for progesterone is in the range between 0.01 and 0.05 ng/ml. If the measured value of progesterone exceeds said selected H* value for progesterone then menarche is anticipated within said known time interval.

In one embodiment, the hormone is 17-hydroxyprogesterone and the selected H* value for 17-hydroxyprogesterone is in the range between 0.01 and 0.10 ng/ml. If the measured value of 17-hydroxyprogesterone exceeds said selected H* value for 17-hydroxyprogesterone then menarche is anticipated within said known time interval.

In one embodiment, the saliva sample is collected by the female. In one embodiment, the female measures the level of at least one hormone in a saliva sample and compares the level of at least one hormone to a selected H* value to determine if menarche is anticipated within said known time interval in her own home.

In one embodiment the steps of a) measuring the level of at least one hormone in a saliva sample from said female; and b) comparing the level of at least one hormone to a selected H* value to determine if menarche is anticipated within said known time interval are completed with the use of an immunoassay. In one embodiment a visual color change of the immunoassay indicates a positive or negative result.

In one embodiment, the known time interval is between 1 to 60 days, or between 1 to 90 days, or between 1 to 45 days, or between 1 to 30 days.

In one embodiment, the saliva sample is obtained by spitting saliva into a tube, or by swabbing saliva from the mouth, or by depositing saliva onto a test strip.

In another embodiment, a method for predicting that the onset of menarche will not occur in a female within a selected time interval is provided. The method comprises a) determining the concentration of at least one hormone selected from the group consisting of 17-bestradiol, testosterone, progesterone, and 17 α hydroxy-progesterone (17-OHP) in a saliva sample from said female; b) determining a Body Mass Index (BMI) value for said female; c) applying the concentration of at least one hormone and the BMI value to a multivariate prediction formula to determine an L value; d) comparing the L value obtained in step (c) to a selected L* value, wherein a L value less than the L* value indicates that menarche is not anticipated within said selected time interval.

In one embodiment the levels of at least two hormones, or at least three hormones or at least four hormones are measured and applied to a multivariate prediction formula for two, three, or four hormones, respectively.

In one embodiment, the L* is selected in a range from −0.05 to +1.60 If said L value is less than the selected L* value then menarche is not anticipated within said selected time interval.

In one embodiment, the L* is selected from a range from 0.90 to 1.20 when the selected time interval is between 1 to 60 days.

In one embodiment, the L* is selected from a range from 1.20 to 1.60 when the selected time interval is between 1 to 90 days.

In one embodiment, the L* is selected from a range from −0.05 to 0.20 when the selected time interval is between 1 to 30 days.

In one embodiment, the L* is selected from a range from 0.10 to 0.40 when the selected time interval is between 1 to 45 days.

In one embodiment, the female is in Tanner Stage III-IV.

In one embodiment, the selected time interval is between 1 to 60 days, or between 1 to 90 days, or between 1 to 45 days, or between 1 to 30 days.

In another embodiment, a method for predicting the onset of menarche in a female within a selected time interval is provided. The method comprises the steps of: a) determining the concentration of at least one hormone selected from the group consisting of 17-bestradiol, testosterone, progesterone, and 17 α hydroxy-progesterone (17-OHP) in a saliva sample from said female; b) determining a Body Mass Index (BMI) value for said female; c) applying the concentration of at least one hormone and the BMI value to a multivariate prediction formula to determine an L value: d) comparing the L value obtained in step (c) to a selected L* value, wherein a L value greater than the L* value indicates that menarche will occur within the selected time interval.

In one embodiment the levels of at least two hormones, or at least three hormones or at least four hormones are measured and applied to a multivariate prediction formula for two, three, or four hormones, respectively.

In one embodiment, the L* is selected in a range from −0.05 to +1.60. If said L value is greater than the selected L* value then menarche will occur within the selected time interval.

In one embodiment, the L* is selected from a range from 0.90 to 1.20 when the selected time interval is between 1 to 60 days.

In one embodiment, the L* is selected from a range from 1.20 to 1.60 when the selected time interval is between 1 to 90 days.

In one embodiment, the L* is selected from a range from −0.05 to 0.20 when the selected time interval is between 1 to 30 days.

In one embodiment, the L* is selected from a range from 0.10 to 0.40 when the selected time interval is between 1 to 45 days.

In one embodiment, the female is in Tanner Stage III-IV.

In one embodiment, the selected time interval is between 1 to 60 days, or between 1 to 90 days, or between 1 to 45 days, or between 1 to 30 days.

In still another embodiment, a kit for predicting that menarche is anticipated in a human female within a known time interval is provided. The kit comprises an indicator responsive to a hormonal level of a hormone in a body fluid, wherein the hormone is selected from the group consisting of testosterone, 17-beta estradiol, progesterone, and 17-hydroxyprogesterone. The indicator provides a positive test result when any one of the following conditions are met: a hormonal level of testosterone exceeds a value selected in a range between about 0.01 and 0.05 ng/ml; a hormonal level of 17-beta estradiol exceeds a value selected in a range between about 0.5 and 10.0 pg/ml; a hormonal level of progesterone exceeds a value selected in a range between about 0.01 and 0.05 ng/ml; a hormonal level of 17-hydroxyprogesterone exceeds a value selected in a range between about 0.01 and 0.10 ng/ml.

In one embodiment the kit is provided for the known time interval between 1 to 60 days, or between 1 to 90 days, or between 1 to 45 days, or between 1 to 30 days.

In one embodiment, the kit is provided for a female in Tanner stage III or IV, and may, for example, comprise a chart specifying how to determine Tanner Stage

In another embodiment, a kit for predicting that menarche is not anticipated in a human female within a known time interval is provided. The kit comprises an indicator responsive to a hormonal level of a hormone in a body fluid, wherein said hormone is selected from the group consisting of testosterone, 17-beta estradiol, progesterone, and 17-hydroxyprogesterone. The indicator provides a positive test result when any one of the following conditions are met: a hormonal level of testosterone does not exceed a value selected in a range between about 0.01 and 0.05 ng/ml; a hormonal level of 17-beta estradiol does not exceed a value selected in a range between about 0.5 and 10.0 pg/ml; a hormonal level of progesterone does not exceed a value selected in a range between about 0.01 and 0.05 ng/ml; a hormonal level of 17-hydroxyprogesterone does not exceed a value selected in a range between about 0.01 and 0.10 ng/ml.

In one embodiment the kit is provided for the known time interval between 1 to 60 days, or between 1 to 90 days, or between 1 to 45 days, or between 1 to 30 days.

In one embodiment, the kit is provided for a female in Tanner stage III or IV, and may, for example, comprise a chart specifying how to determine Tanner Stage.

### BRIEF DESCRIPTION OF THE DRAWINGS

FIGS. 1A and 1B show example logistic regression formulas and calculated threshold cutoff values, H* and L*, for a 60 day prediction of menarche. FIG. 1A, column 1 is labeled “predictors” and indicates either no predictors or the predictors of Body Mass Index (BMI), 17-β estradiol (E2), testosterone (TEST), progesterone (PROG), 17 hydroxy-progesterone (OHP) and combinations thereof. FIG. 1A, column 2, labeled receiver operator characteristic (ROC) Area shows the area under the ROC curve for the univariate and multivariate predictor logistic regression formulas, with its standard error of estimate to the right (column 3, SE, a “plus-or-minus” index indicating the precision attached the reported value of ROC area). Threshold values are displayed in the next two columns: column 4, H* values labeled “1-vbl cutoff”; and column 5, L* values labeled “formula cutoff.” Columns 6, 8 and 10 show the false positive rate (FPR), the positive predictive value (PPV), and the negative predictive value (NPV), respectively. Standard errors are shown to the right of each FPR, PPV, and NPV in columns 7, 9 and 11 labeled “SE.” Logistic regression formulas constructed for the various predictors are shown in FIG. 1B.

FIGS. 2A and 2B show example logistic formulas and calculated threshold cutoff values, H* and L*, for a 30 day prediction of menarche. The figures are laid out as described in the legend for FIGS. 1A and 1B.

FIGS. 3A and 3B show example logistic formulas and calculated threshold cutoff values, H* and L*, for a 45 day prediction of menarche. The figures are laid out as described in the legend for FIGS. 1A and 1B.

FIGS. 4A and 4B show example logistic formulas and calculated threshold cutoff values, H* and L*, for a 90 day prediction of menarche. The figures are laid out as described in the legend for FIGS. 1A and 1B.

FIG. 5 shows a graphical representation of the negative predictive values (NPV) for the time interval of 1 to 60 days for various univariate and multivariate prediction models described in Example 1. The negative predictive values, depicted on the Y-axis, indicate the probability that a girl who is predicted not to reach menarche within 60 days will not do so. The univariate and multivariate predictors on the X-axis. Using the cut off values described in FIG. 1, the negative predictive values for each model depicted, with the exception of BMI, is above 90%.

FIG. 6 shows a graphical representation of the positive predictive values (PPV) for the time interval of 1 to 60 days for various univariate and multivariate prediction models described in Example 1. The positive predictive values, depicted on the Y-axis, indicate the probability that a girl who is predicted to reach menarche within 60 days will actually do so. The univariate and multivariate predictors on the X-axis. Using the cut off values described in FIG. 1, the positive predictive values for each model depicted, with the exception of BMI, is about 50%.

FIG. 7 shows a graphical representation of the negative predictive values (NPV) for the time interval of 1 to 30 days for various univariate and multivariate prediction models described in Example 1. The negative predictive values, depicted on the Y-axis, indicate the probability that a girl who is predicted not to reach menarche within 30 days will not do so. The univariate and multivariate predictors on the X-axis. Using the cut off values described in FIG. 1, the negative predictive values for each model depicted is above 90%.

FIG. 8 shows a graphical representation of the positive predictive values (PPV) for the time interval of 1 to 30 days for various univariate and multivariate prediction models described in Example 1. The positive predictive values, depicted on the Y-axis, indicate the probability that a girl who is predicted to reach menarche within 30 days will actually do so. The univariate and multivariate predictors on the X-axis. Using the cut off values described in FIG. 1, the positive predictive values for each model depicted is about 10-20%.

FIG. 9 shows a graphical representation of the negative predictive values (NPV) for the time interval of 1 to 45 days for various univariate and multivariate prediction models described in Example 1. The negative predictive values, depicted on the Y-axis, indicate the probability that a girl who is predicted not to reach menarche within 45 days will not do so. The univariate and multivariate predictors on the X-axis. Using the cut off values described in FIG. 1, the negative predictive values for each model depicted, with the exception of BMI alone, is above 90%.

FIG. 10 shows a graphical representation of the positive predictive values (PPV) for the time interval of 1 to 45 days for various univariate and multivariate prediction models described in Example 1. The positive predictive values, depicted on the Y-axis, indicate the probability that a girl who is predicted to reach menarche within 45 days will actually do so. The univariate and multivariate predictors on the X-axis. Using the cut off values described in FIG. 1, the positive predictive values for each model depicted is about 20-30%.

FIG. 11 shows a graphical representation of the negative predictive values (NPV) for the time interval of 1 to 90 days for various univariate and multivariate prediction models described in Example 1. The negative predictive values, depicted on the Y-axis, indicate the probability that a girl who is predicted not to reach menarche within 90 days will not do so. The univariate and multivariate predictors on the X-axis. Using the cut off values described in FIG. 1, the negative predictive values for the models ranges from about 60%-90%.

FIG. 12 shows a graphical representation of the positive predictive values (PPV) for the time interval of 1 to 90 days for various univariate and multivariate prediction models described in Example 1. The positive predictive values, depicted on the Y-axis, indicate the probability that a girl who is predicted to reach menarche within 90 days will actually do so. The univariate and multivariate predictors on the X-axis. Using the cut off values described in FIG. 1, the positive predictive values for the depicted models ranges from about 30-65%.

FIG. 13 shows a ROC curved fitted for the multivariate logistic regression formula for the selected period of 1 to 60 days before the onset of menarche and the predictor variables are 17-beta estradiol (E2), testosterone (TEST) and body mass index (BMI). The summary statistics of the ROC curved is also provided, indicating the predictive strength of the said multivariate logistic regression formula.

FIG. 14 shows the graphical experimental data of L values calculated using the multivariate logistic regression formulae for the predictor variables 17-beta estradiol (E2), testosterone (TEST) and body mass index (BMI). The X-axis represents the period of 1 to 150 days before the onset of menarche and the time of onset of menarche is set at 0 time. The Y-axis represents the L values. The L* value of 1.025 is selected from FIG. 13's ROC curve where the sensitivity is 80% and is specificity is also 80%.

FIG. 15 shows a schematic diagram of a method of the invention for determining that the level of a hormone in saliva is greater than a predetermined threshold level using a simple one-step analytical strategy.

FIG. 16 shows a schematic diagram showing the interpretation of the results obtained using the simple one-step analytical strategy shown in FIG. 15.

FIG. 17 shows a schematic diagram showing a simple one-step analytical strategy where the levels of four hormones are determined simultaneously using four different test strips.

FIG. 18 shows a schematic diagram showing a simple one-step analytical strategy where the levels of three hormones are determined simultaneously on the same membrane and test strip.

FIG. 19 shows a schematic diagram showing an alternate version of the method of the invention using a simple one-step analytical strategy.

FIG. 20 shows a schematic diagram showing the interpretation of the results obtained using the simple one-step analytical strategy shown in FIG. 19.

FIG. 21 shows a schematic diagram showing an alternate simple one-step analytical strategy where the levels of four hormones are determined simultaneously using four different test strips.

FIG. 22 shows a schematic diagram showing an alternate simple one-step analytical strategy where the levels of three hormones can be determined simultaneously on the same membrane in a single dipstick test strip.

FIG. 23 shows a ROC curved fitted for the univariate logistic regression formula for the selected period of 1 to 60 days before the onset of menarche and the predictor variable is 17-beta estradiol (E2). The summary statistics of the ROC curved is also provided, indicating the predictive strength of the said univariate logistic regression formula.

FIG. 24 shows the graphical experimental data of H values calculated using the univariate logistic regression formulae for the predictor variables 17-beta estradiol. The X-axis represents the period of 1 to 150 days before the onset of menarche and the time of onset of menarche is set at 0 time. The Y-axis represents the H values. The H* value of 8.52 pg/ml is selected from FIG. 23's ROC curve where the sensitivity is 75% and is specificity is also 75%.

### DETAILED DESCRIPTION OF THE INVENTION

Embodiments of the invention provide methods and diagnostic kits for reliably predicting that the onset of menarche will or will not, occur in girls and young women within a known time interval, e.g. 1 to 30 days, 1 to 45 days, 1 to 60 days, or 1 to 90 days.

As used herein, the term “menarche” refers to a females first menstrual period.

We have established that useful prediction formulas can be constructed for specified time intervals, including but not limited to, 1 to 30 days, 1 to 45 days, 1 to 60 days and 1 to 90 days, by applying both univariate and multiple logistic regression analysis to data sets reflecting the levels of salivary hormones present in females before, and at the onset, of menarche.

In one embodiment according to the invention, predetermined threshold levels of hormones, H*, are determined using univariate logistic regression analysis and these H* levels are used as the threshold level of hormone which serves as a predictor as to whether or not menarche will occur in a female (e.g. a female in Tanner Stage I-V) within a known time interval including, but not limited to, 1 to 30 days, 1 to 45 days, 1 to 60 days, and 1 to 90 days. If a girl's salivary hormone level is below the threshold level, H*, it is anticipated that menarche will not occur within the interval. Alternatively, if a if a girl's salivary hormone level is above a threshold level, H*, it is anticipated that menarche will occur within the selected time interval.

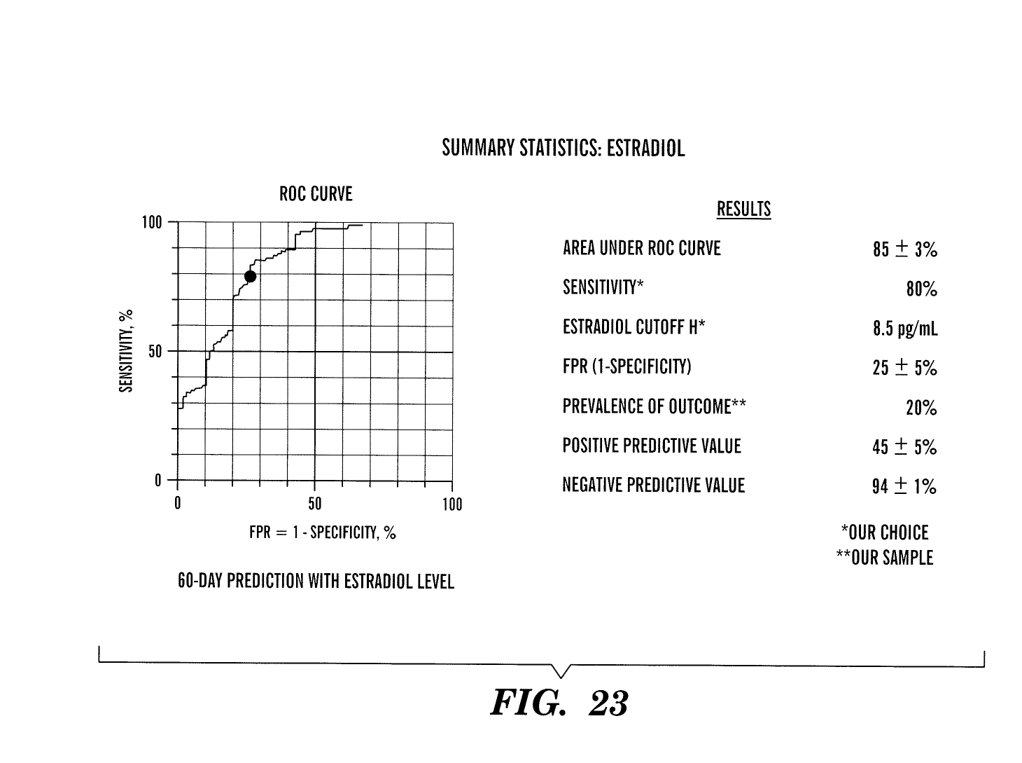

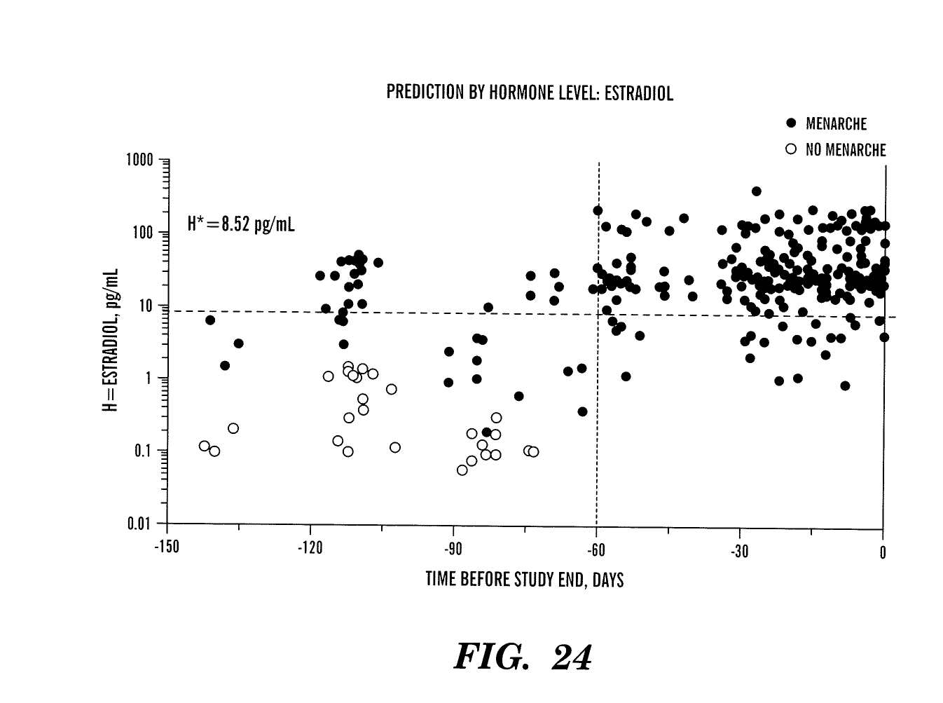

As used herein, “a predetermined threshold amount of hormone” or “H*” refers to the amount of hormone determined to be a threshold level of hormone for a female where salivary hormone levels below a threshold amount, H*, indicate that menarche in said female will not occur and salivary hormone levels above a H* indicate that menarche will occur within a selected time interval. Using means known to those skilled in the art, a predetermined threshold amount of hormone is determined for a desired interval and a desired outcome by applying univatriate logistic regression analysis to a data set of premenarchal hormone levels determined for each hormone in the selected “known time interval.” The fitted logistic model takes the following form in the case of a single hormone: Logit [probability(menarche within interval)]=constant+coefficient×log hormone. The predetermined threshold hormone level is selected to be high or low enough to guarantee a desired sensitivity, e.g. a sensitivity of 80% or greater. H* can be selected with the aid of a receiver operating characteristic curve (a ROC curve) analysis which plots the true positive rate (sensitivity) versus the false positive rate (1-specificity) (FIG. 23). One would select a threshold level of hormone that renders the highest degree of sensitivity while keeping the false positive rate to a minimum, e.g. a false positive rate under about 0.4, or about 0.3. H* can be selected to render varied probabilities of accuracy for the selected outcome (whether menarche will, or will not occur), e.g. 45-50%, 50-55%, 55-60%, 60-65%, 65-70%, 70-75%, 75-80%, 80-90%, 90-95%, or 95-100%. For example, the H* value of 8.52 pg/ml (FIG. 24) is selected from the ROC curve (FIG. 23) for the fitted univariate logistic regression formula where 17-beta estradiol is the sole predictor variable and a specified time interval of 1 to 60 days such that the sensitivity is about 75% and is specificity is also 75%.

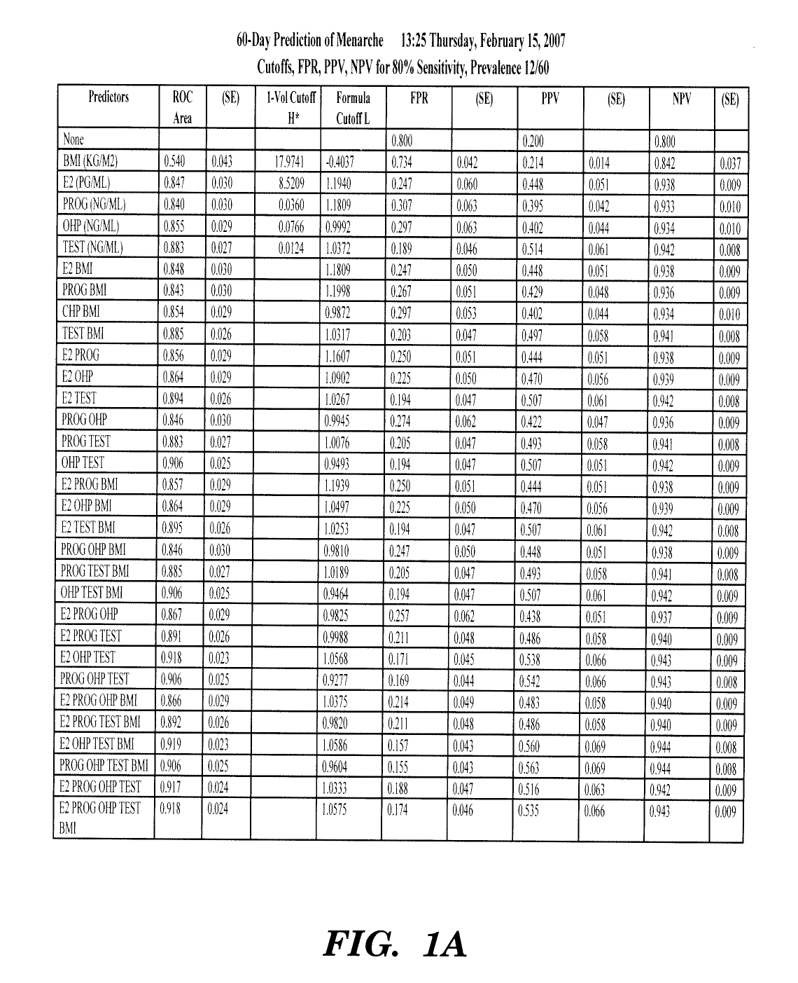

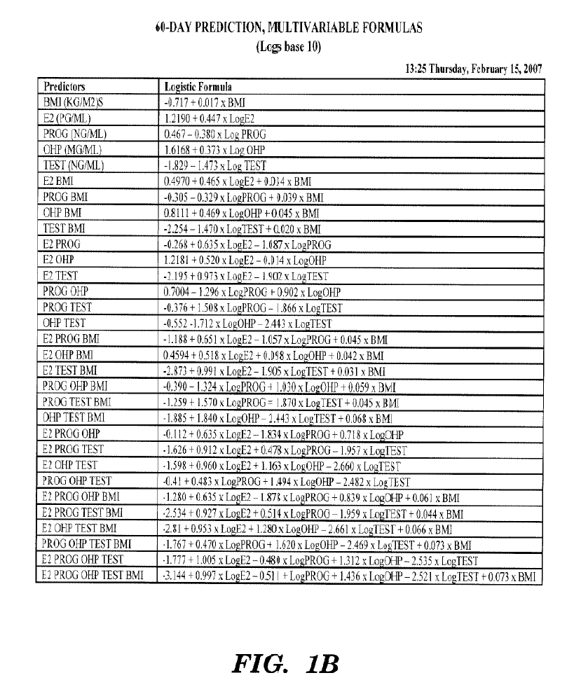

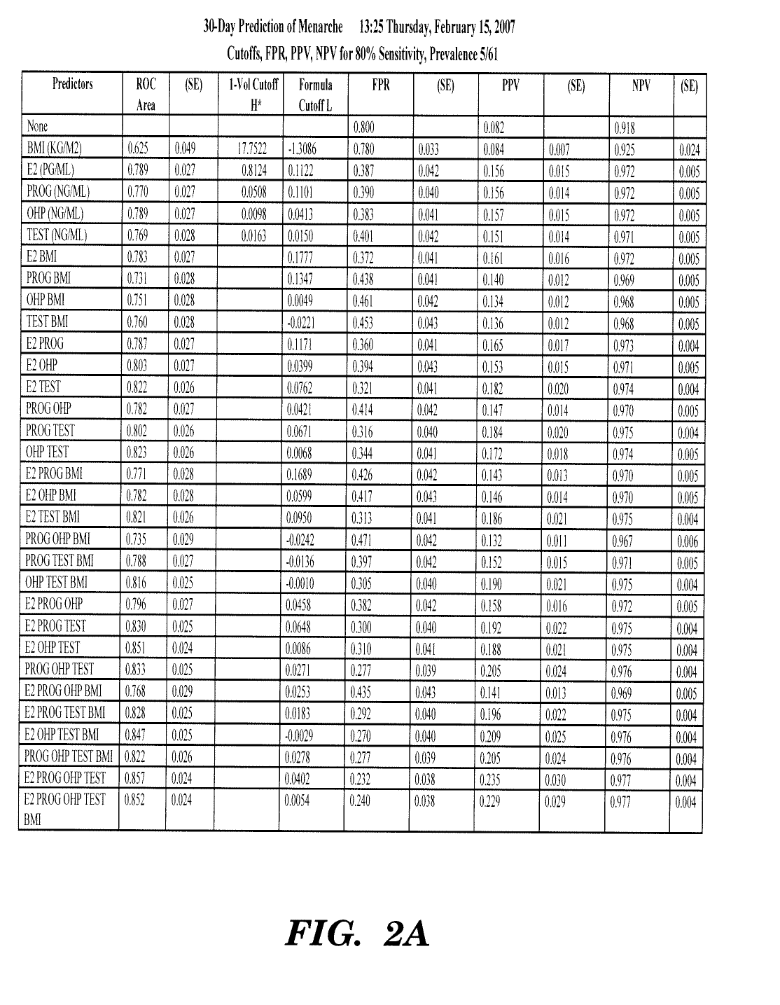

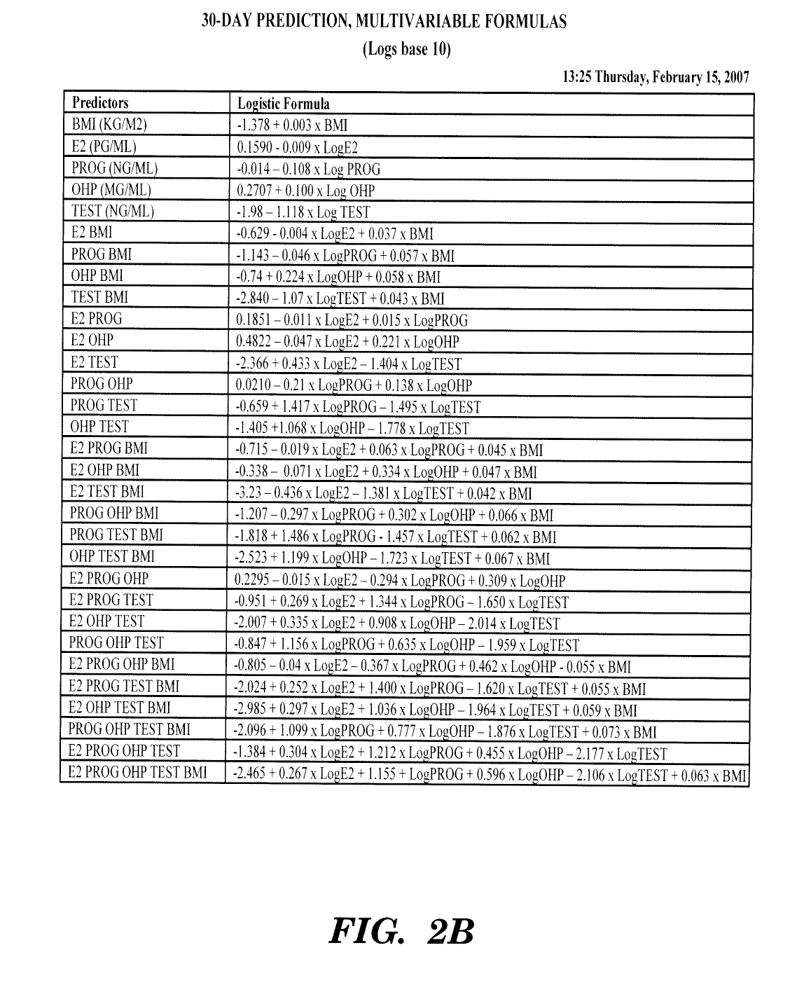

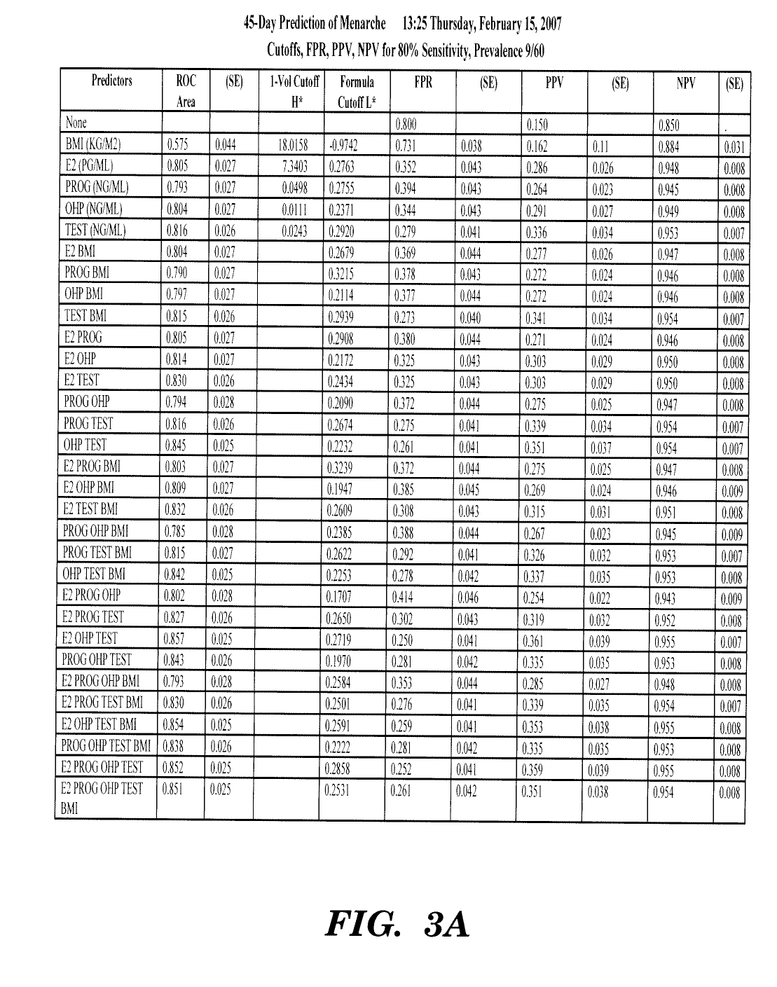

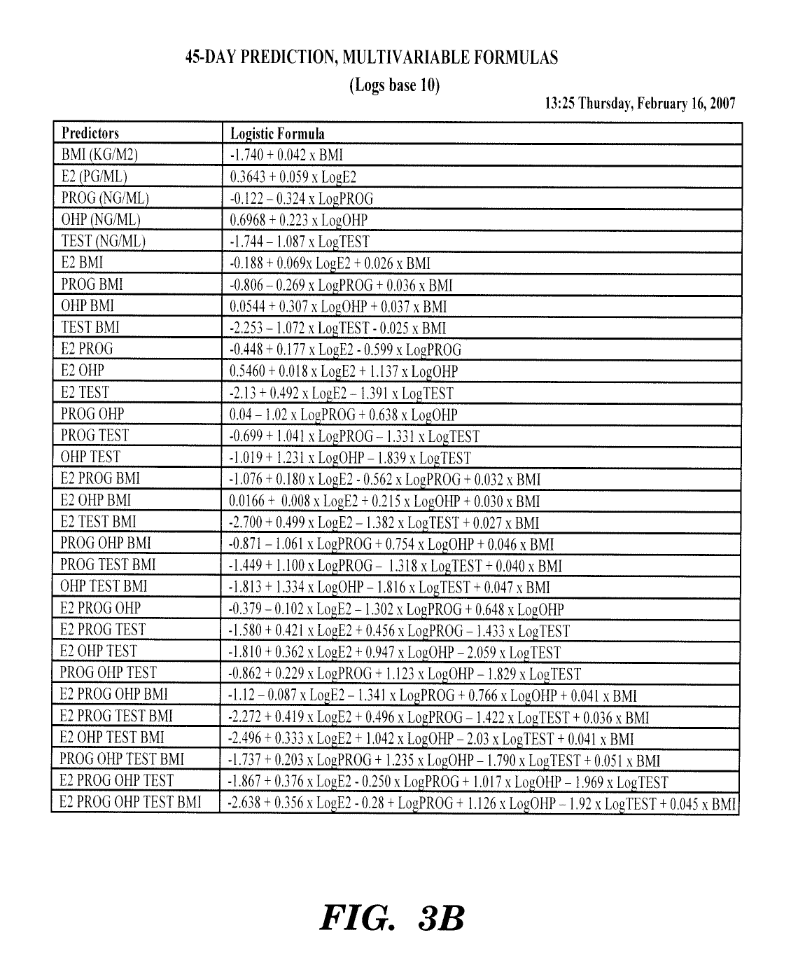

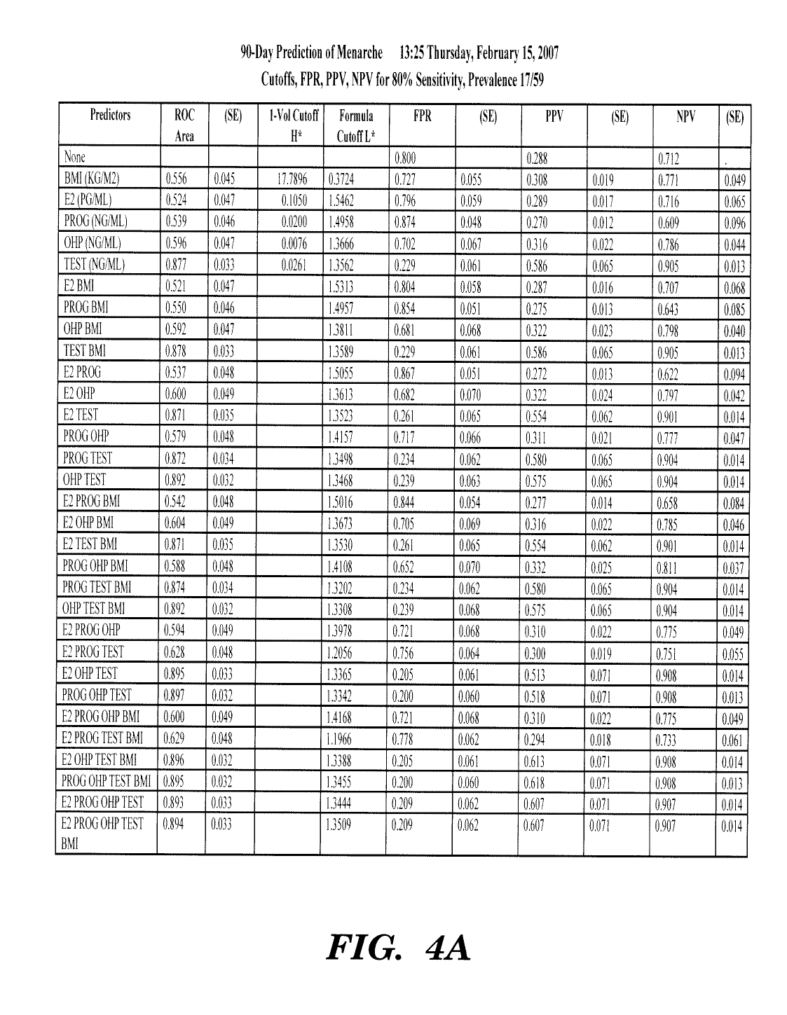

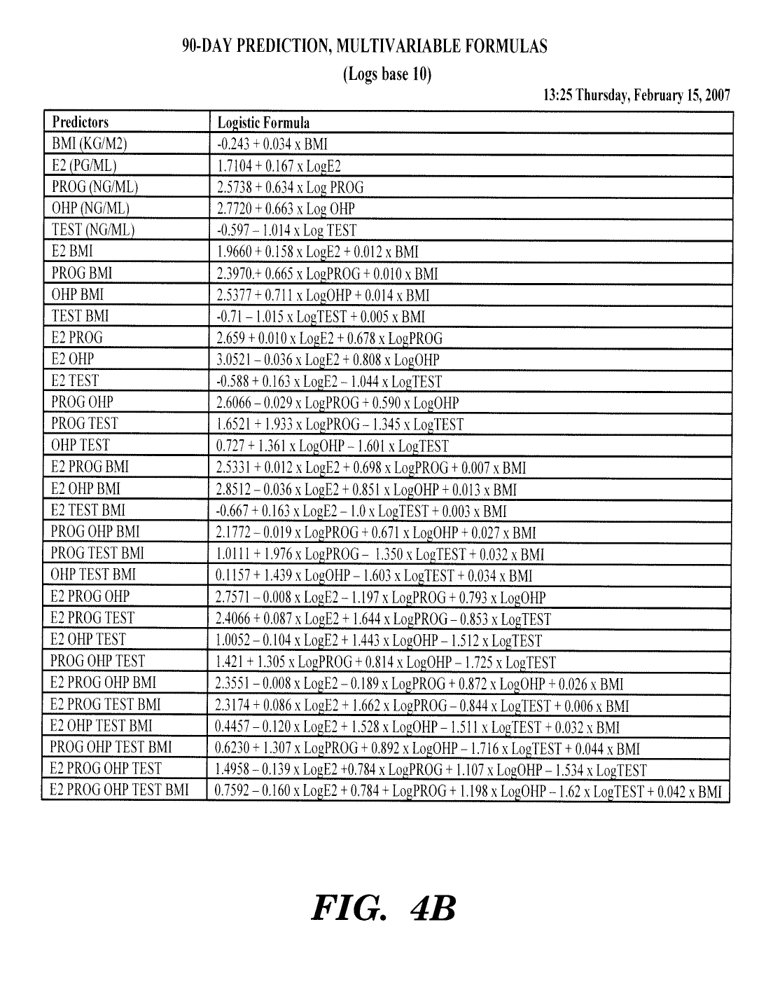

As an example, predetermined threshold amounts of hormone, H*, were selected using logistic regression analysis of weekly salivary hormonal levels in a sample set of 63 Tanner Stage III-IV females for the intervals of 1 to 30 days, 1 to 45 days, 1 to 60 days, and 1 to 90 days (see FIGS. 1-4, and Example 1). FIG. 1A, column 4, shows the predetermined threshold hormone levels, H*, selected for 17-β estradiol (E2, pg/ml), testosterone (TEST, ng/ml), progesterone (PROG, ng/ml), and 17 [symbol] hydroxy-progesterone (OHP, ng/ml) such that levels below the threshold indicate that there is about 93-94% probability (NPV values from about 0.93 to about 0.94, column 10) that the Tanner Stage III-IV female will not reach menarche within 60 days. These H* threshold levels were about 8.5 g/ml for 17-β estradiol, about 12.4 pg/ml for testosterone, about 36.0 pg/ml for progesterone, and about 76.6 pg/ml for 17 α hydroxy-progesterone (17-OHP). Threshold levels were also determined for these hormones using the selected time intervals of 1 to 30 days (FIG. 2A), 1 to 45 days (FIG. 3A), and 1 to 90 days (FIG. 4A) and these levels were correlated to the probabilities that menarche will (PPV) or will not occur (NPV) within the selected time interval (see FIGS. 1A-4A, columns 8 and 10).

Accordingly, in one embodiment, a method of predicting that menarche is not anticipated within a specified time interval in a human female is provided. The method involves measuring the level of at least one hormone in a saliva sample from a female and comparing the measured level to a selected H* value to determine if menarche is not anticipated within the time interval. A salivary hormone level below H* indicates menarche will not occur within the specified time interval.

In one embodiment of the invention, ranges of H* values for the hormones testosterone, 17-beta estradiol, progesterone, and 17-α hydroxyprogesterone for the specified time interval are provided. The H* value for testosterone can be selected from a range between 0.01 and 0.05 ng/ml and the H* value of 17-beta estradiol is selected from a range between 0.5 and 10.0 pg/ml. Similarly the H* values for progesterone is selected from a range between 0.01 and 0.05 ng/ml and the H* value for 17-hydroxyprogesterone is be selected from a range between 0.01 and 0.10 ng/ml.

In one embodiment, a method of predicting that menarche is not anticipated within 1 to 60 days in a human female in Tanner Stage III-IV is provided. The method involves measuring the level of at least one hormone (e.g. 17-β estradiol, testosterone, progesterone, and/or 17 [symbol] hydroxy-progesterone) in a saliva sample from the female and comparing the measured level to a selected H* value to determine if menarche is not anticipated within the time interval. A level of hormone below the selected H* value indicates that menarche will not occur with in 60 days. In this embodiment, the selected H* threshold amount of hormone can range from 5.00 pg/ml-15.00 pg/ml for 17-beta-estradiol, from 8.00 pg/ml-18 pg/ml for testosterone, from 30 pg/ml-40 pg/ml for progesterone, and from 70 pg/ml-80 pg/ml for 17 hydroxy-progesterone (17-OHP). In one embodiment, H* is about 8.5 pg/ml 17 beta-estradiol, about 0.012 ng/ml testosterone, about 0.036 ng/ml progesterone, and about 0.076 ng/ml 17 hydroxy-progesterone (17-OHP).

In another embodiment, a method of predicting that menarche is anticipated in a human female within a known time interval is provided. The method comprises measuring the level of at least one hormone in a saliva sample from a female and comparing the level hormone to a selected H* value to determine if menarche is anticipated within said known time interval. A salivary hormone level above the selected H* indicates that menarche will occur within the specified time interval.

In one embodiment, the salivary hormone to be measured hormone is selected from the group consisting of testosterone, 17-beta estradiol, progesterone, 17-α hydroxyprogesterone, and combinations thereof.

In one embodiment of the invention, ranges of H* values for the hormones testosterone, 17-beta estradiol, progesterone, and 17-α hydroxyprogesterone for the specified time interval are provided. The H* value for testosterone is selected from a range between 0.01 and 0.05 ng/ml and the H* value of 17-beta estradiol is selected from a range between 0.5 and 10.0 pg/ml. Similarly the H* values for progesterone is selected from a range between 0.01 and 0.05 ng/ml and the H* value for 17-hydroxyprogesterone is selected from a range between 0.01 and 0.10 ng/ml.

In one embodiment, the female to be tested for the onset of menarche is in Tanner Stage III or IV.

As used herein, the terms “Tanner Stage I”, “Tanner Stage II”, “Tanner Stage III”, “Tanner Stage IV” and “Tanner Stage V” refer to the female Tanner stages of breast development and pubic hair development. Tanner stages of breast development include Tanner Stage I, a preadolescent, the nipple elevates; Tanner Stage II, the breast bud develops and the breast tissue and nipple arise as a single mound of tissue; Tanner Stage III, the single mound enlarges; Tanner Stage IV, a secondary mound develops, with the nipple and areola projecting above the breast tissue; Tanner Stage V, the areola regresses to form a smooth contour with the rest of the breast tissue. Tanner stages of pubic hair include Tanner Stage I, no pubic hair; Tanner Stage II, a small amount of long, downy hair with slight pigmentation on the labia majora; Tanner Stage III, hair becomes more coarse and curly, and begins to extend laterally; Tanner Stage IV, adult-like hair quality, extending across pubis but sparing medial thighs; and Tanner Stage V, hair extends to medial surface of the thighs.

Saliva samples can be collected by any means known to those skilled in the art, e.g. by spitting saliva into a tube, by swabbing saliva from the mouth, or by depositing saliva onto a test strip.

Any hormone present in saliva that is useful to predict menarche in a female can be measured and used to predict menarche using methods of the invention. Hormonal levels in the saliva sample can be measured by any means known to those skilled in the art. In one embodiment, the levels of salivary hormone/s is/are measured using an immunoassay. Such an immunoassay can provide a positive or negative result as an indication as to whether or not menarche will occur. Means for measuring the levels of salivary hormones are described in more detail herein under the subheading Measuring levels of Salivary Hormone.

In particular embodiments of the invention, a female can collect her own saliva sample and measure the levels of hormones in her saliva in the privacy of her own home. For example using a home test kit.

In another aspect according to the invention, it has been determined that multivariate prediction formulas can been constructed for varied time intervals using multiple logistic regression analysis with incidence of menarche as the dichotomous dependent variable and combinations of hormone levels and Body Mass Index as independent variables. Such multivariate prediction formulas can reliably determine whether or not menarche will occur in a female within a specified time interval. As a proof of principle example, we have constructed multivariate prediction formulas for selected time intervals of 1 to 30 days, 1 to 45 days, 1 to 60 days and 1 to 90 days, by applying multiple logistic regression analysis to Body Mass Index (BMI) data and weekly levels of salivary hormone data from a population of pre-menarchal females in Tanner Stage III or greater, and whose BMI is greater than the 5^{th} percentile for age. The levels of 17-β estradiol (E2), testosterone (TEST), progesterone (PROG), and 17 α hydroxyl-progesterone (OHP) were tested (See Example 1 and FIGS. 1-4).

As used herein, the term Body Mass Index” or “BMI” refers to a measure of body fat based on height and weight. It is calculated by divided a person's weight (measured in kilograms) by height in meters squared. (BMI=kg/m²) in the metric system. In the English system, weight is in pounds and height is in feet and inches.

Means for applying multiple logistic regression analysis to data are well known to those skilled in the art. The fitted logistic model takes the following form in the case of a single hormone and BMI: Logit[Probability(menarche within interval)]=L=constant+coefficient×log hormone×BMI. The formula for BMI and two hormones, is Logit[Probability(menarche within interval)]=L=constant+coefficient×BMI+coefficient×log hormone #1+coefficient×log hormone #2. The formula for BMI and three hormones, is Logit[Probability(menarche within interval)]=L=constant+coefficient×BMI+coefficient×log hormone #1+coefficient×log hormone #2+coefficient×log hormone #3. While the formula for or BMI and four hormones is Logit[Probability(menarche within interval)]=L=constant+coefficient×BMI+coefficient×log hormone #1+coefficient×log hormone #2+coefficient×log hormone #3+coefficient×log hormone #4.

In practice, in the case of multiple predictors, the prediction is made by a rule of the following form: a) specify a threshold logit value, which is L*; b) if a girl's hormone levels and BMI entered into the logistic formula produce a L value above the threshold, predict menarche within the interval; if below, predict no menarche.

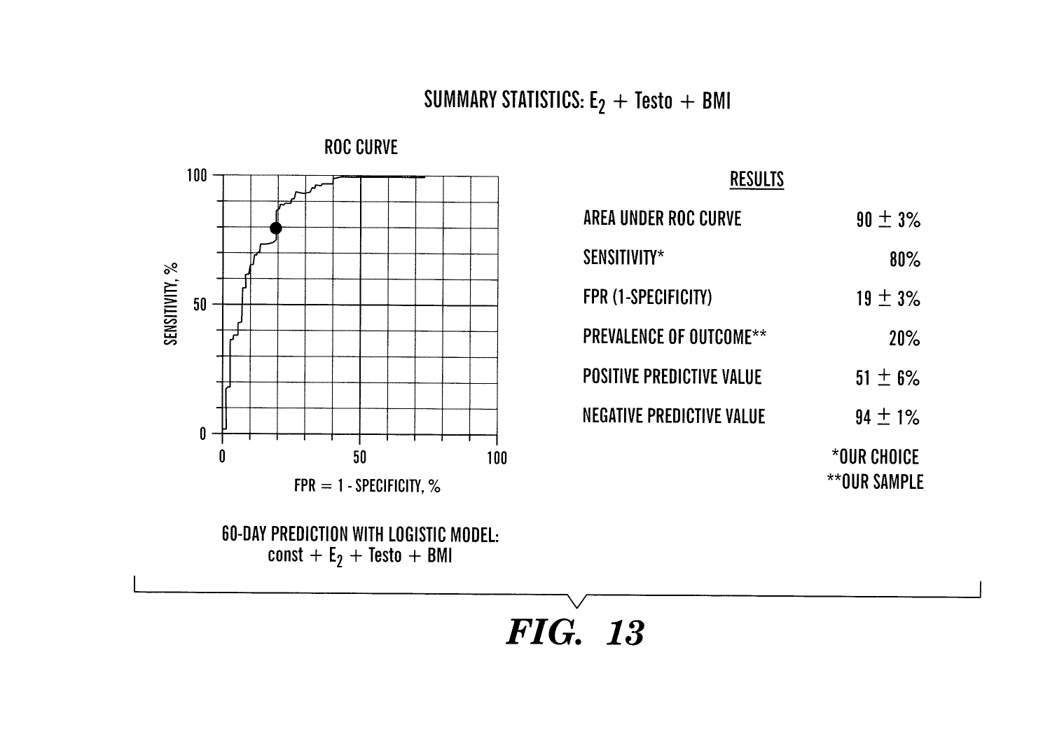

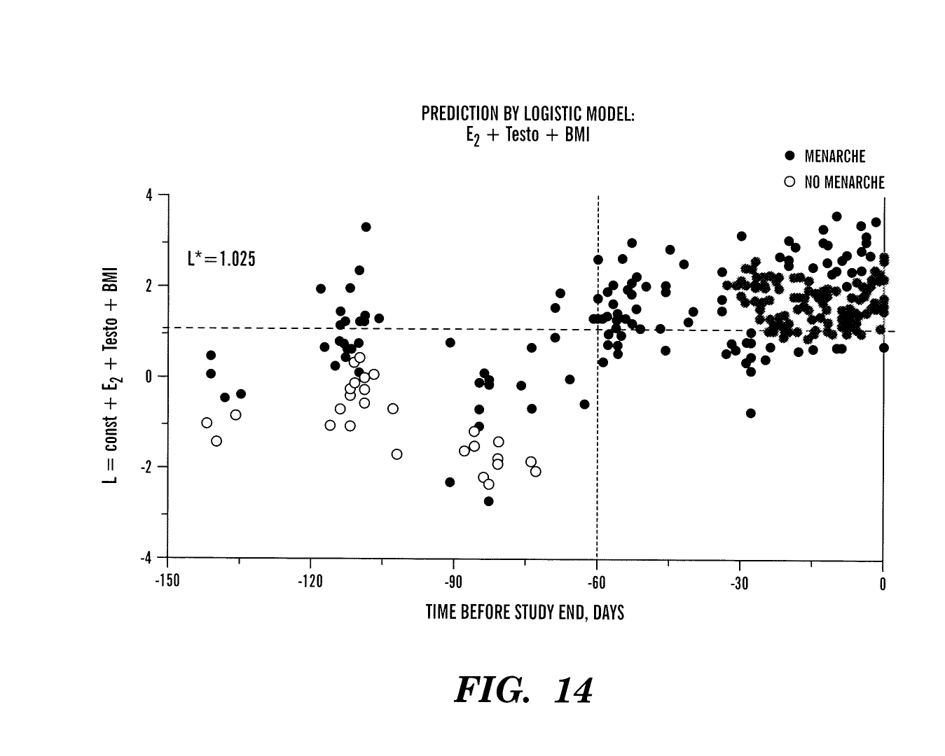

The L* value is selected to provide the desired sensitivity and specificity. It can be selected from a receiver operator characteristic (ROC) curve that has a c-statistic or area under the ROC curve of at least 0.85 which is a measure of the predictive strength of a multi-variant logistic regression, the prediction formula. A ROC curve of a multi-variant logistic regression that gives a c-statistic ranging from about 0.85 to 0.99 is indication of the strong predictive strength of that multi-variant logistic regression. The L* value is selected from a range on the ROC curve that provides the highest degree of sensitivity while keeping the false positive rate to a minimum. For example, at least 65% sensitivity and at least 65% specificity, e.g., if the assay is to predict that the female will reach menarche in a known time interval, the sensitivity is the probability that a girl who is going to reach menarche will be correctly predicted to do so and the specificity is the probability that a girl who is not going to reach menarche will be correctly predicted not to do so. A range of sensitivity may be around 75-95% with a specificity of 75-95%. For example, a prediction formula for three variables: 17 (3-estradiol, testosterone, and BMI, and for menarche onset between 1-60 days has a good c-statistic or area under the ROC curve of 0.90 (FIG. 13). The L* value that will provide 80% sensitivity and 80% specificity to the prediction is 1.025 (FIG. 14).

As an example, prediction formulas to obtain L and L* values have been determined using a population of 63 females in Tanner Stage III-IV, for the following variable combinations (predictors) at intervals of 1 to 30 days, 1 to 45 days, 1 to 60 days and 1 to 90 days; the variable combinations are: BMI and 17-β estradiol (E2); BMI and testosterone (TEST); BMI and progesterone (PROG); BMI and 17 hydroxy-progesterone (OHP); 17-β estradiol (E2) and testosterone; 17-β estradiol (E2) and progesterone (PROG); 17-β estradiol (E2) and 17 hydroxy-progesterone (OHP); progesterone and testosterone; progesterone and 17 hydroxy-progesterone; 17 hydroxy-progesterone (17-OHP) and testosterone; 17-β estradiol, BMI and testosterone; 17-β estradiol, BMI, and progesterone; 17-β estradiol, BMI, and 17 hydroxy-progesterone (17-OHP); progesterone, BMI, and testosterone; progesterone, BMI, and 17 hydroxy-progesterone (17-OHP); 17 hydroxy-progesterone (17-OHP), BMI and testosterone; 17-β estradiol, progesterone and 17 hydroxy-progesterone (17-OHP); 17-β estradiol, progesterone and testosterone; 17-β estradiol, 17 hydroxy-progesterone (17-OHP) and testosterone; progesterone, 17 hydroxy-progesterone (17-OHP) and testosterone; 17-β estradiol, BMI, progesterone and 17 hydroxy-progesterone (17-OHP); 17-β estradiol, BMI, progesterone and testosterone; 17-β estradiol, BMI, 17 hydroxy-progesterone (17-OHP) and testosterone; progesterone, BMI, 17 hydroxy-progesterone (17-OHP) and testosterone; 17-β estradiol, testosterone, progesterone, and 17 hydroxy-progesterone (17-OHP); and 17-β estradiol, BMI, testosterone, progesterone, and 17 hydroxy-progesterone (17-OHP).

Multivariate prediction formulas and L* threshold values for the prediction of menarche within the intervals of 1 to 30 days, 1 to 45 days, 1 to 60 days and 1 to 90 days for the above combinations are indicated in FIGS. 1-4. L* is indicated by the formula cut off values listed in column 5 of FIGS. 1A, 2A, 3A and 4A. The multivariate logistic formulas to obtain L values for each selected time interval are listed in FIGS. 1B, 2B, 3B, and 4B.

In one embodiment, a method for predicting that the onset of menarche will not occur within a selected time interval in a female subject is provided. The method comprises a) determining the level of at least one hormone selected from the group consisting of 17-β estradiol, testosterone, progesterone, and 17 α hydroxy-progesterone (17-OHP) in a saliva sample from said female subject; b) determining a Body Mass Index (BMI) value for said female; c) applying the level of at least one hormone and the BMI value to a multivariate prediction formula to determine an L value; and d) comparing the L value obtained in step (c) to a selected L* level, where a L value less than L* indicates that menarche will not occur in the selected time interval in said female. In one embodiment the selected time interval is 1 to 30 days. In one embodiment the selected time interval is 1 to 45 days. In one embodiment the selected time interval is 1 to 60 days. In one embodiment the selected time interval is 1 to 90 days. In one embodiment the female is in Tanner stage III or IV.

In one embodiment, the L* values is selected from a range between −0.05 to +1.60 and said range is dependent on the time interval specified. In a preferred embodiment, the L* is selected from a range from 0.90 to 1.20 when the selected time interval is between 1 to 60 days. In another preferred embodiment, the L* is selected from a range from 1.20 to 1.60 when the selected time interval is between 1 to 90 days. In yet another preferred embodiment, the L* is selected from a range from −0.05 to 0.20 when the selected time interval is between 1 to 30 days. In another preferred embodiment, the L* is selected from a range from 0.10 to 0.40 when the selected time interval is between 1 to 45 days.

In one embodiment, a method for predicting that the onset of menarche will occur within a selected time interval in a female subject is provided. The method comprises a) determining the level of at least one hormone selected from the group consisting of 17-β estradiol, testosterone, progesterone, and 17 α hydroxy-progesterone (17-OHP) in a saliva sample from said female subject; b) determining a Body Mass Index (BMI) value for said female; c) applying the level of at least one hormone and the BMI value to a multivariate prediction formula to determine an L value; and d) comparing the L value obtained in step (c) to a selected L* level, where a L value greater than L* indicates that menarche will occur in the selected time interval in said female. In one embodiment the selected time interval is 1 to 30 days. In one embodiment the selected time interval is 1 to 45 days. In one embodiment the selected time interval is 1 to 60 days. In one embodiment the selected time interval is 1 to 90 days. In one embodiment the female is in Tanner stage III or IV.

In one embodiment, the L* values is selected from a range between −0.05 to +1.60 and said range is dependent on the time interval specified. In a preferred embodiment, the L* is selected from a range from 0.90 to 1.20 when the selected time interval is between 1 to 60 days. In another preferred embodiment, the L* is selected from a range from 1.20 to 1.60 when the selected time interval is between 1 to 90 days. In yet another preferred embodiment, the L* is selected from a range from −0.05 to 0.20 when the selected time interval is between 1 to 30 days. In another preferred embodiment, the L* is selected from a range from 0.10 to 0.40 when the selected time interval is between 1 to 45 days.

Measuring Levels of Salivary Hormone

In embodiments of the invention, the level of hormone in saliva is measured to obtain a determination of whether or not menarche will occur. The salivary hormone levels can be measured using any assay known to those skilled in the art, including, but not limited to, Enzyme-Linked Immunosorbent Assay (ELISA), immunoprecipitation assays, radioimmunoassay, mass spectrometry, Western Blotting, and via dipsticks using conventional technology.

For purposes of comparison, the level of hormone in the saliva should be measured in the same manner as the predetermined threshold level is measured. For example, the levels of hormone can be represented in arbitrary units dependent upon the assay used to measure the levels of hormone, e.g., the intensity of the signal from the detectable label can correspond to the amount of hormone present (e.g. as determined by eye, densitometry, an ELISA plate reader, a luminometer, or a scintillation counter).

The levels of hormone present in saliva samples can be determined using a ligand that specifically binds to the hormone, e.g, a synthetic peptide, chemical, or antibody. The ligand is preferably detectably labeled.

In one embodiment of the invention, immunoassays using antibodies are used to measure the levels of hormone in saliva. As used herein, the term “antibody” is intended to include immunoglobulin molecules and immunologically active determinants of immunoglobulin molecules, e.g., molecules that contain an antigen binding site which specifically binds (immunoreacts with) to the hormone to be measured. The term “antibody” is intended to include whole antibodies, e.g., of any isotype (IgG, IgA, IgM, IgE, etc), and includes fragments thereof which are also specifically reactive with the hormone to be measured, e.g. 17-β estradiol, testosterone, progesterone, and/or 17 hydroxy-progesterone (17-OHP)). Antibodies can be fragmented using conventional techniques. Thus, the term “antibody” includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's may be covalently or non-covalently linked to form antibodies having two or more binding sites. Thus, “antibody” includes polyclonal, monoclonal, or other purified preparations of antibodies and recombinant antibodies. The term “antibody” is further intended to include humanized antibodies, bispecific antibodies, and chimeric molecules having at least one antigen binding determinant derived from an antibody molecule. In one embodiment, the antibody is detectably labeled.

Antibodies to the hormones can be generated using methods known to those skilled in the art. Alternatively, commercially available antibodies can be used. Antibodies to 17-β estradiol, testosterone, progesterone, and/or 17 hydroxy-progesterone (17-OHP) are commercially available.

As used herein “detectably labeled”, includes antibodies that are labeled by a measurable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, HIS, or biotin.

In the diagnostic methods of the invention that use an antibody for the detection of hormone levels, the level of hormone present in the saliva samples correlates to the intensity of the signal emitted from the detectably labeled antibody.

In one embodiment, the antibody is detectably labeled by linking the antibody to an enzyme. The enzyme, in turn, when exposed to it's substrate, will react with the substrate in such a manner as to produce a chemical moiety which can be detected, for example, by spectrophotometric, fluorometric, or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. Chemiluminescence is another method that can be used to detect an antibody.

Detection may also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by audoradiography. Isotopes which are particularly useful for the purpose of the present invention are ³H, ¹³¹I, ³⁵S, ¹⁴C, and preferably ¹²⁵I.

It is also possible to label an antibody with a fluorescent compound. When the fluorescently labelled antibody is exposed to light of the proper wave length, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

In one embodiment the levels of hormone in saliva are detected by ELISA assay. There are different forms of ELISA which are well known to those skilled in the art, e.g. standard ELISA, competitive ELISA, and sandwich ELISA. The standard techniques for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In one embodiment, the levels of salivary hormone are determined by contacting a saliva sample with a first antibody that specifically binds to a hormone to be measured under conditions permitting formation of a complex between the antibody and said hormone (e.g. 17-β estradiol, testosterone, progesterone, and 17 [symbol] hydroxy-progesterone (17-OHP)). The amount of complex formed is then measured as a measure of the level of the hormone, and the amount of complex formed is compared to the amount of complex formed between the first antibody and a predetermined threshold amount of the hormone. A level below the threshold amount of hormone indicates that menarche will not occur, while a level above the threshold amount of hormone indicates that menarche will occur.

In one embodiment, the first antibody is detectably labeled. Detectably labeling the first antibody is appropriate for use, for example, in standard ELISA assays where hormone is absorbed to an ELISA plate, or in Western Blot analysis, or certain dipstick analyses.

In one embodiment, the first antibody is immobilized on a solid support, for example, when using a “Sandwich Elisa” or a dipstick analysis, then the amount of complex formed can measured by detecting binding of a second antibody that specifically binds to the hormone (e.g. 17-β estradiol, testosterone, progesterone, and 17 hydroxy-progesterone (17-OHP)) under conditions permitting formation of a complex between the second antibody and the hormone, wherein the second antibody does not substantially cross-react with the first antibody, and wherein the second antibody is detectably labeled.

Any solid support can be used, including but not limited to, nitrocellulose, solid organic polymers, such as polystyrene, or laminated dipstcks such as described in U.S. Pat. No. 5,550,375.

In one embodiment, the levels of two hormones defining a first and a second hormone, are measured using at least two antibodies specific to each hormone to be measured. Each antibody specifically reacts either the first hormone or the second hormone to be measured while not substantially cross-reacting with the other hormones to be measured.

In one embodiment, the levels of three hormones defining a first hormone, a second hormone, and a third hormone are measured using at least three antibodies specific to each hormone to be measured, wherein each antibody specifically reacts either the first hormone, the second hormone, or the third hormone to be measured while not substantially cross-reacting with the other hormones to be measured.

In one embodiment, the levels of four hormones defining a first, a second, a third hormone and a fourth hormone, are measured using at least four antibodies specific to each hormone to be measured, wherein each antibody specifically reacts either the first hormone, the second hormone, the third hormone, or the fourth hormone to be measured while not substantially cross-reacting with the other hormones to be measured.

In one embodiment, the hormones are selected from the group consisting of 17-β estradiol, testosterone, progesterone, and 17- [symbol] hydroxy-progesterone (17-OHP).

The use of “dip sticks” or test strips and other solid supports have been described in the art in the context of an immunoassay for a number of antigens. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. Additional “dip stick” diagnostics appropriate for use in embodiments of the invention are described in Examples III and IV.

Embodiments of the invention further provide for diagnostic kits and products of manufacture comprising the diagnostic kits. The kits can comprise a means for predicting that menarche is anticipated in a human female, they can comprise a means for predicting that menarche is not anticipated in a human female within a known time interval, or they can comprise both. The known time interval can be, for example, between 1 to 60 days, or between 1 to 90 days, or between 1 to 30 days, or between 1 to 45 days. Other known time intervals can be used.

In one embodiment, the kit comprises an indicator responsive to a hormonal level of a hormone in a body fluid, wherein the hormone is selected from the group consisting of testosterone, 17-beta estradiol, progesterone, and 17-hydroxyprogesterone. The indicator provides a positive test result when any one of the following conditions are met: a hormonal level of testosterone exceeds a value selected in a range between about 0.01 and 0.05 ng/ml; a hormonal level of 17-beta estradiol exceeds a value selected in a range between about 0.5 and 10.0 pg/ml; a hormonal level of progesterone exceeds a value selected in a range between about 0.01 and 0.05 ng/ml; a hormonal level of 17-hydroxyprogesterone exceeds a value selected in a range between about 0.01 and 0.10 ng/ml.

In on embodiment the kit comprises an indicator responsive to a hormonal level of a hormone in a body fluid, wherein said hormone is selected from the group consisting of testosterone, 17-beta estradiol, progesterone, and 17-hydroxyprogesterone. The indicator provides a positive test result when any one of the following conditions are met: a hormonal level of testosterone does not exceed a value selected in a range between about 0.01 and 0.05 ng/ml; a hormonal level of 17-beta estradiol does not exceed a value selected in a range between about 0.5 and 10 pg/ml; a hormonal level of progesterone does not exceed a value selected in a range between about 0.01 and 0.05 ng/ml; a hormonal level of 17-hydroxyprogesterone does not exceed a value selected in a range between about 0.01 and 0.10 ng/ml.

In one embodiment, the body fluid can be saliva, tears, urine, sweat, blood plasma, or vaginal secretion. The kits can further comprise cups or tubes, or any other collection device for sample collection of the body fluids.

In one embodiment, the kits further comprise charts, for example describing the Five Tanner Stages of development, describing the interpretation of test results, or describing how to calculate body mass index.

An article of manufacture comprising a package of tampons or menstrual pads and a kit comprising a means for predicting the onset of menarche for a female subject is also contemplated.

This invention is further illustrated by the following examples which should not be construed as limiting. The contents of all references, patents, and patent applications cited throughout this application, as well as the figures and table are incorporated herein by reference.

### EXAMPLES

### Example I

Levels of salivary 17-β estradiol, testosterone, progesterone, and 17-hydroxyprogesterone (17-OHP) and BMI, alone or in combination, can predict menarche in healthy premenarcheal girls.

### Materials and Methods

We enrolled 63 pre-menarcheal females ages 9-15 yr, with BMI >5th percentile for age and Tanner Stage ≧III for both axillary/pubic hair and breast development. Weekly saliva samples were collected for 12 months or until first menses, and monthly height and weight were recorded. Salivary 17-β estradiol, testosterone, progesterone, and 17-OHP concentrations were determined by Enzyme-Linked ImmunoSorbent Assay. Multiple logistic regression analysis was used to construct prediction formulas for menarche within 30, 45, 60, and 90 days of a given saliva sample, with the four hormone levels and most recent BMI as predictors.

### Procedure

Participants collected weekly saliva samples in a pre-labeled 2 ml eppendorf tube using ParafilmM® to induce salivation and straws to reduce contamination. Subjects were instructed to choose a convenient day and time to collect the weekly saliva samples. If they missed the chosen day and time participants were instructed to collect the saliva sample as soon as possible. The date of sample collection was recorded on the eppendorf tube label and the diary card. The time of collection was not documented. Participants used a diary to record the dates samples were collected, adverse events, medications used, and presence of any breast tenderness or swelling and vaginal discharge or bleeding. Dated samples were stored in the home freezer and transported to the monthly clinic visit using a Styrofoam cooler and ice packs. At monthly clinic visits height, weight, and body mass index were recorded, diary cards were reviewed, saliva samples were collected and new supplies were dispensed. Subject's remained in the study for 12 months or until first menses, which ever came first. During the termination visit subjects' vital signs, height, weight and body mass index were measured.

### Specimen Handling and Selection

The saliva samples were de-identified and stored at −70 degrees Celsius. Samples collected 0, 1, 2, 3, 4, 8, and 16 weeks+/−3 days prior to the first day of their menstrual cycle or study termination date (if subject did not reach menarche) were assayed using Enzyme-Linked ImmunoSorbent Assay for salivary 17-β estradiol, testosterone, progesterone, and 17-OHP concentrations. For those subjects who missed a sample or reached menarche prior to 16 weeks from study initiation, as many samples as were available were assayed. Samples were also later assayed from weeks 12 and 20 for patients whose samples had not reached the one-year expiration mark.

### Analysis

On completion of data collection, we had measurements from 63 subjects available for analysis, with a total of approximately 200 monthly BMI measurements and 350-400 weekly salivary levels of the four hormones. We fixed four potential intervals at which to construct formulas for prediction of menarche: 30, 45, 60, and 90 days.

Among the 63 participants, 61 were observed long enough to determine whether or not they reached menarche within a 30-day interval. The incidence of menarche was 6/51, or 8%, and the number of hormone measurements available for prediction (i.e., preceding menarche or end-of-study by at least 30 days) was 259. The corresponding figures for the other intervals were as follows: for 45 days, incidence 9/60=15% with 257 measurements; for 60 days, incidence 12/60=20% with 246 measurements; and for 90 days, 17/59=29% with 236 measurements 90 days.

To construct a prediction formula, we conducted multiple logistic regression analysis with incidence of menarche as the dichotomous dependent variable and some combination of hormone levels and BMI as independent variables. When BMI was analyzed alone, we used only the monthly measurements. When BMI was combined with hormones, we used the most recent BMI measurement for those dates at which only saliva was collected. Hormone values, having skewed distributions, were log-transformed for analysis to reduce the destabilizing influence of extreme values.

The fitted logistic model takes the following form in the case of a single hormone:

Logit[Prob(menarche within interval)]=constant+coefficient×log hormone. If more than one predictor is employed, then the model is a multiple logistic equation. With BMI and two hormones, for example, the formula is Logit[Prob(menarche within interval)]=constant+coefficient×BMI+coefficient×log hormone #1+coefficient×log hormone #2. In practice, the prediction is made by a rule of the following form: a) Specify a threshold hormone concentration. If a girl's hormone level is above the threshold, predict menarche within the interval; if below, predict no menarche.

In the case of multiple predictors used jointly, the rule is slightly more complex: a) Ssecify a threshold logit value b) If a girl's hormone levels and/or BMI, entered into the logistic formula, produce a value above the threshold, predict menarche within the interval; if below, predict no menarche.

The strength of prediction can be summarized by several different statistical parameters, all derived from the fitted model. The most general is the area under the receiver operating characteristic (ROC) curve, an index between 0.5 and 1. A value of 0.5 is no better than random chance; a value of 1 is perfect prediction.

Other commonly cited summary statistics include the sensitivity (probability that a girl who is going to reach menarche will be correctly predicted to do so) and specificity (probability that a girl who is not going to reach menarche will be correctly predicted not to do so). The false positive rate (FPR) is the complement of specificity, i.e., probability that a girl who is not going to reach menarche within the interval will be incorrectly predicted to do so. These parameters are under the user's control to a certain degree. One may choose the threshold hormone level or logit value to be high or low enough to guarantee a desired sensitivity. The specificity and FPR will then be determined by the ROC curve. Conversely, one may choose specificity (equivalently, choose FPR) and let sensitivity be determined by the ROC curve. These parameters trade off; higher sensitivity produces higher FPR and lower specificity.

Two other commonly cited summary statistics are the positive predictive value (PPV, probability that a girl who is predicted to reach menarche within the interval will actually do so) and negative predictive value (NPV, probability that a girl who is predicted not to reach menarche within the interval will not do so). These two parameters, being conditioned on the result of the hormones and/or BMI, depend not only on the quality of the prediction formula but also on the prior probability of menarche in the population to which they relate. For our purposes, the germane population is girls similar to those enrolled in the study, i.e., age 13-18 and Tanner stage III-IV. The probabilities of menarche are those cited earlier, ranging from 8% within 30 days to 29% within 90 days.

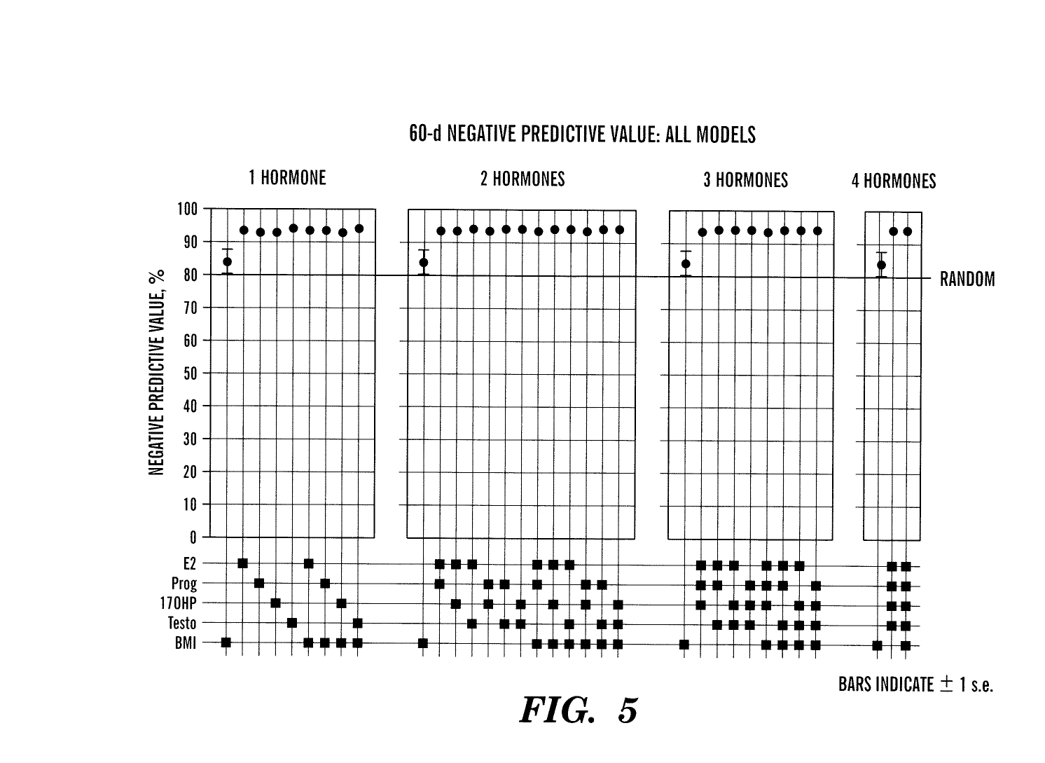

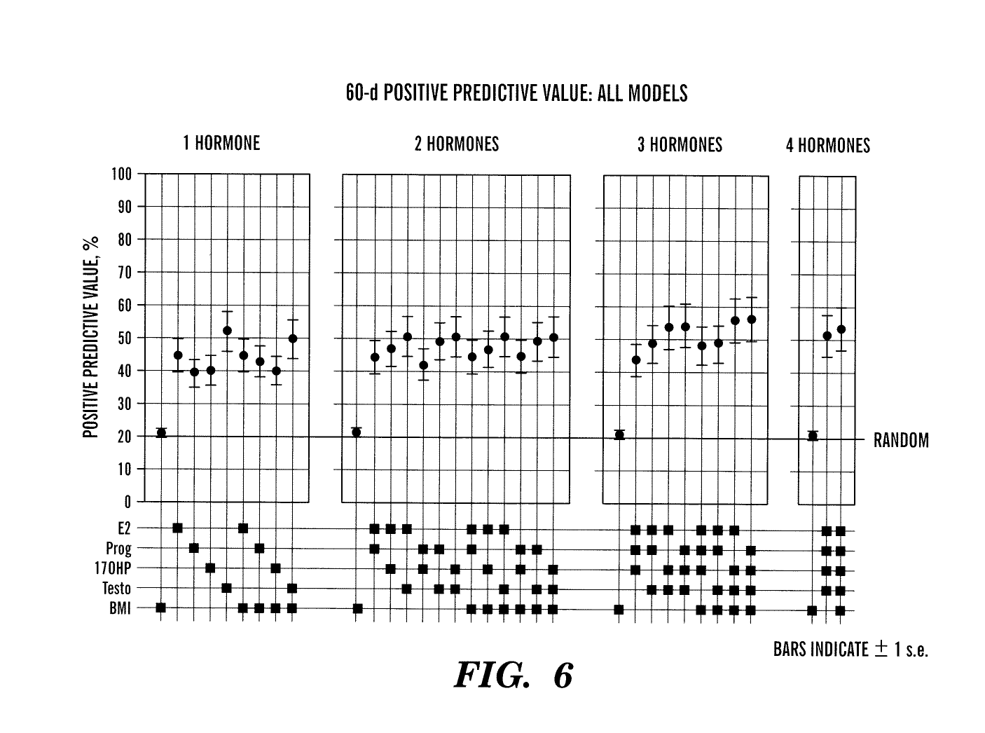

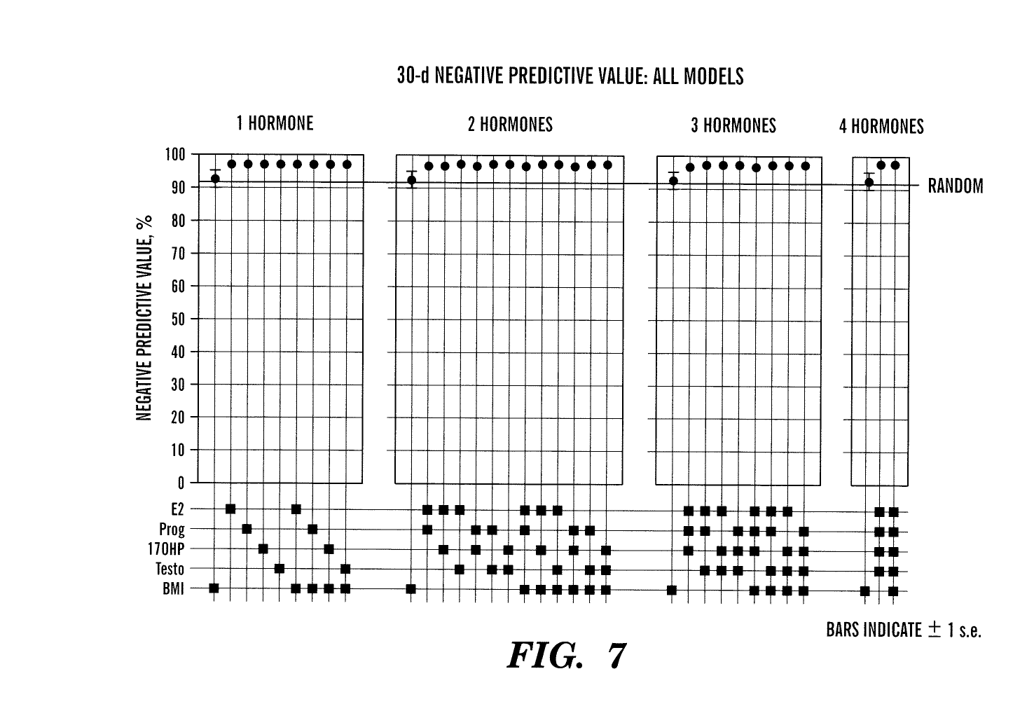

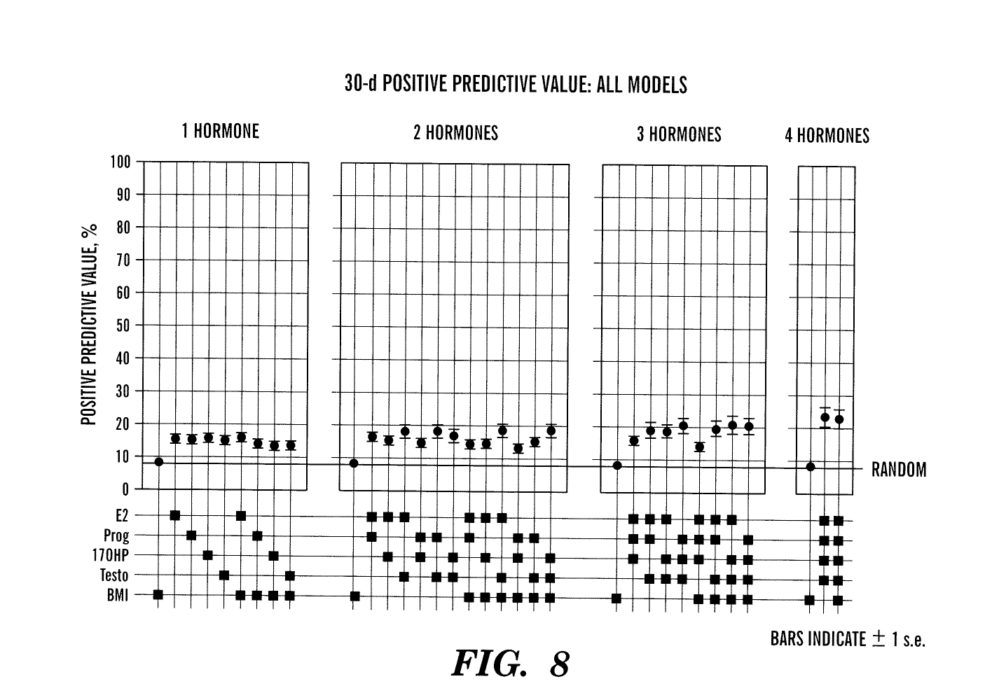

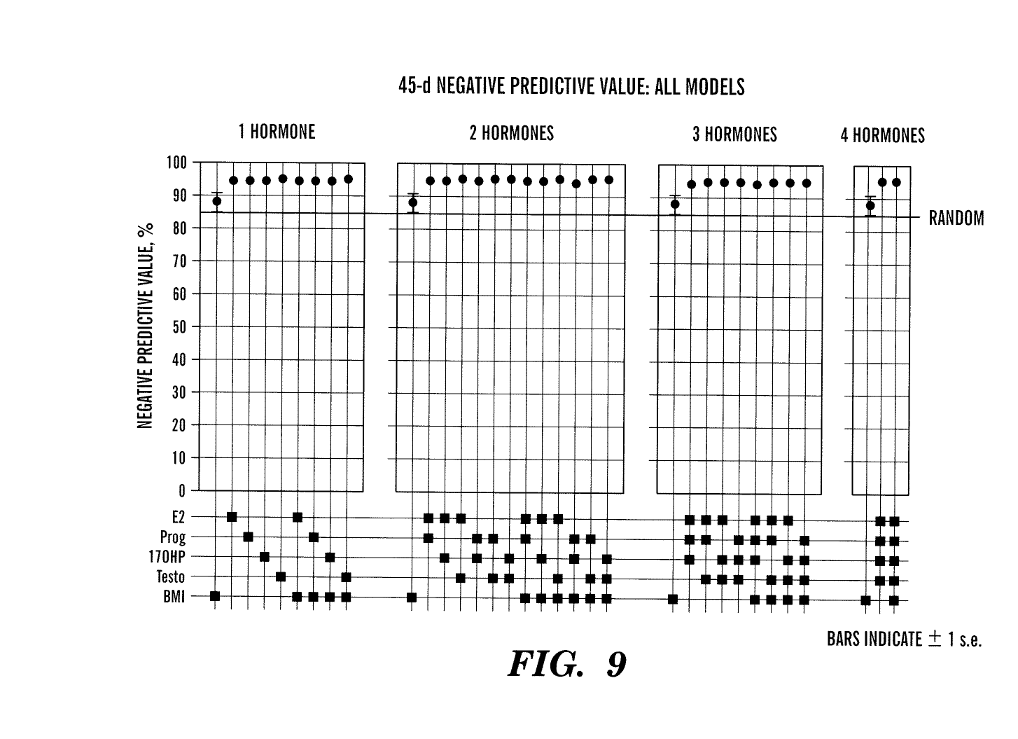

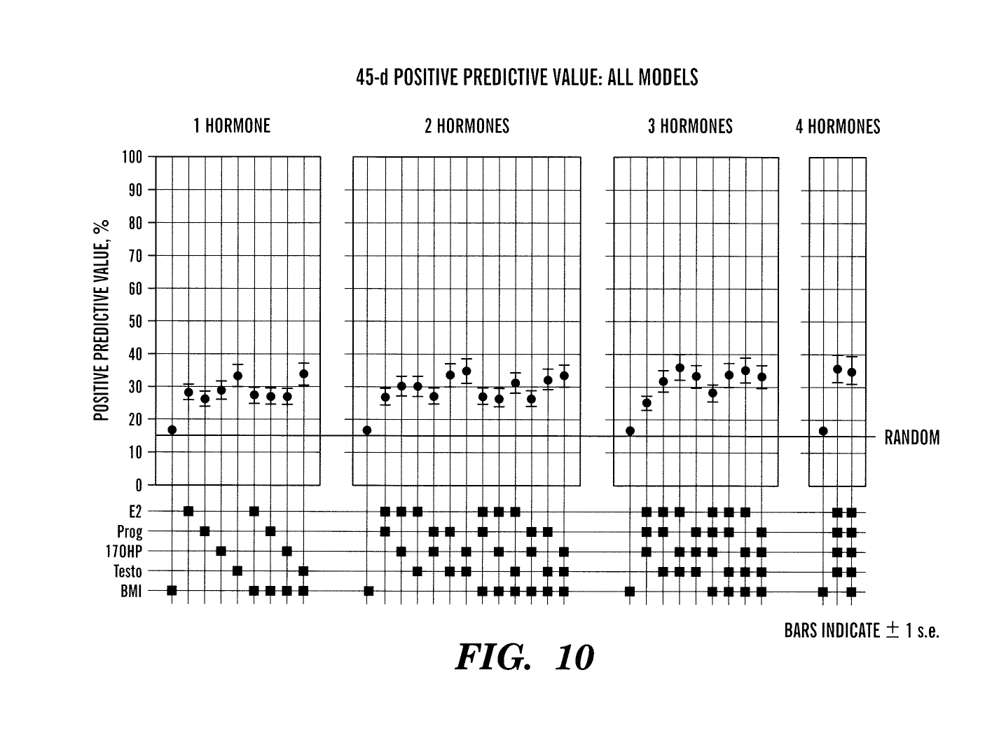

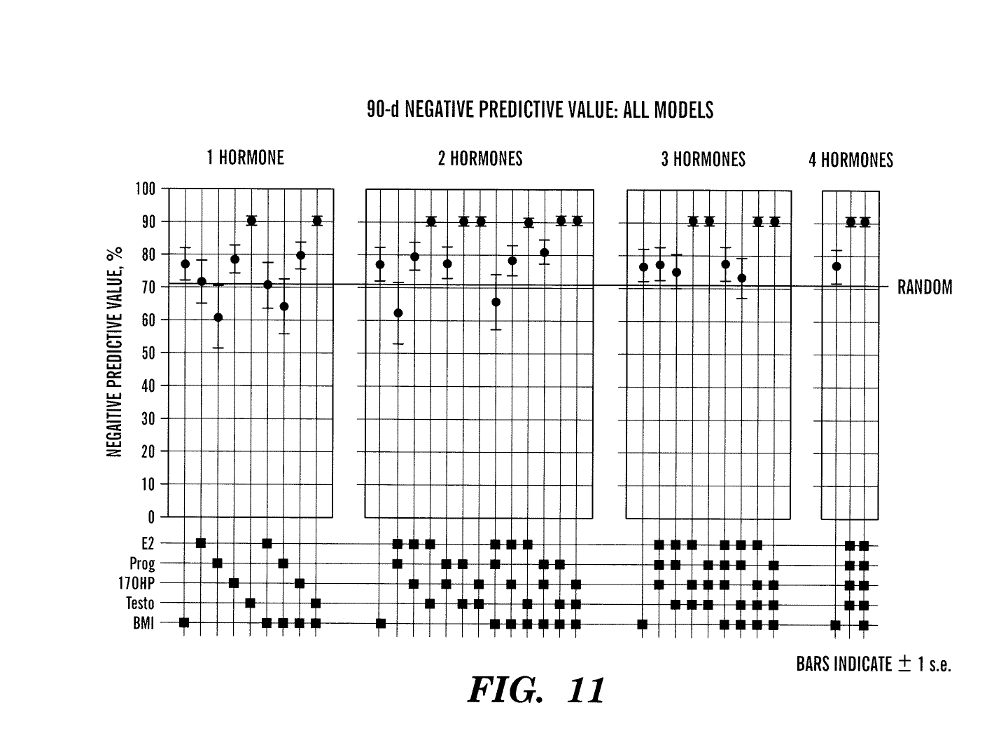

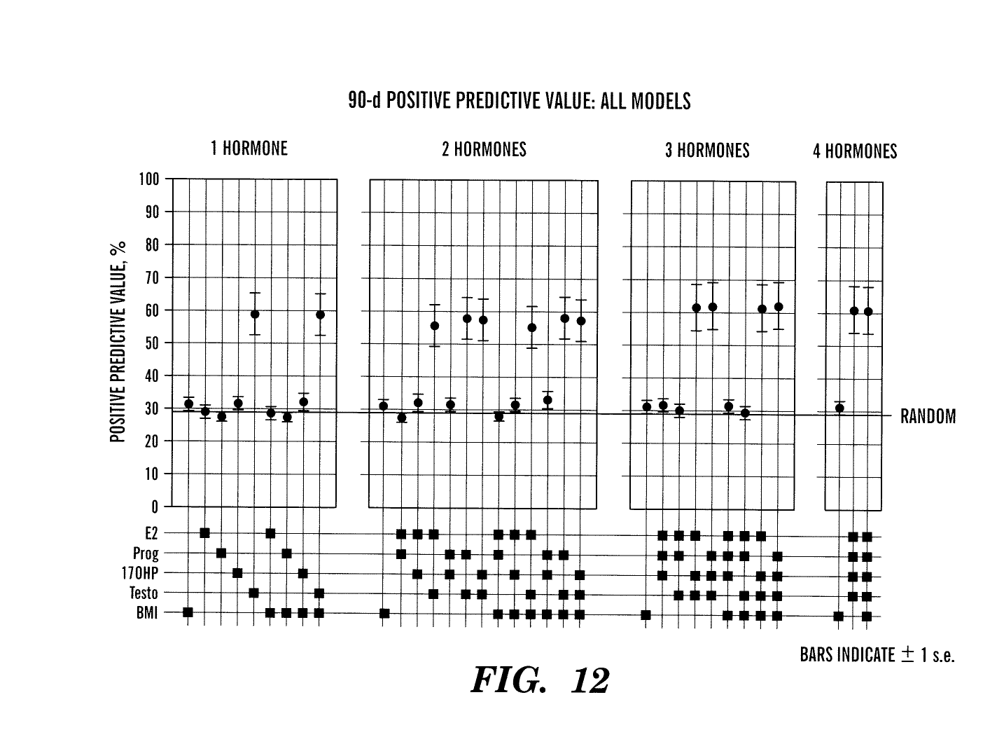

We applied this methodology for each of the four prediction intervals and each possible combination of one or more salivary hormone measurements, with and without including BMI. We also assessed BMI alone as a predictor. The results are tabulated in FIGS. 1-4, with one figure comprising the results for a particular prediction interval. The 32 possible combinations of BMI and hormones run down the left margin of the figures, column 1 labeled “predictors”. Column 2 of the figures labeled “ROC Area” show the ROC area for the model, with its standard error of estimate to the right (column 3, SE, a “plus-or-minus” index indicating the precision attached the reported value of ROC area). We chose cutoff values for each logistic formula to produce 80% sensitivity, which would be a minimum for a reasonable predictive instrument in this setting. The cutoffs are displayed in the next two columns: column 4, H* values labeled “1-vbl cutoff”; and column 5, L* values labeled “formula cutoff”. Columns 6, 8 and 10 show the false positive rate (FPR), the positive predictive value (PPV), and the negative predictive value (NPV) respectively. Each of the latter also carries a standard error, columns 7, 9 and 11 labeled “SE”. In FIGS. 1B, 2B, 3B, and 4B are displayed the fitted logistic formulas for each predictor or set of predictors. Graphical summaries of PPV and NPV are also attached for each prediction interval, FIGS. 5-12.

### Results

Of 63 girls enrolled, 41 were Tanner Stage III, 20 stage IV and 2 stage V for breast development at entry; 38 were stage III and 25 stage IV for pubic hair. Fifty-five girls completed protocol, 43 with menarche and 12 without. Eight subjects dropped out or were lost to follow-up. Of 60 subjects observed >60 days, 12 (20%) reached menarche within that interval. One prediction formula showed sensitivity 80%, specificity 83%, positive predictive value (PPV) 54%, and negative predictive value (NPV) 94% for prediction of menarche within 60 days. This formula can be used to reliably determine that menarche will not occur within 60 days in girls using measured salivary hormone levels and BMI of premenarcheal girls in Tanner Stage III-IV. Such a high NPV may be especially useful in clinical situations where it would be advantageous to accurately predict the timing of menarche such as in cancer patients or anxious girls going to summer camp.

### Example II

Provided are two examples of how the prediction formulas derived in Example I would be used for individual girls.

A) Suppose we propose to use estradiol (E2) to predict menarche within 60 days. The cutoff value is 8.5 pg/ml (see FIG. 1A, E2, column H*, and FIG. 24). Two girls are determined to be eligible for testing, both being of appropriate age and Tanner stage (Tanner Stage III-IV). Girl #1 shows salivary estradiol of 10.5 pg/ml. Because this value is above the cutoff, we predict she will reach menarche within 60 days. The chance that this will actually come to pass is 44.8% (PPV), see FIG. 1A, column 8, predictor E2. Girl #2 shows salivary estradiol of 6.1 pg/ml, which is below the cutoff. We therefore predict she will not reach menarche within 60 days. The chance that this prediction will hold is 93.8% (NPV), see FIG. 1A, column 10, predictor E2.

(B) Suppose we want to use 17-hydroxyprogesterone (OHP) and BMI to predict menarche within 60 days. The logistic formula is 0.8111+0.469×log(OHP)+0.045×BMI, (see FIG. 1B, predictors OHP and BMI, row 8, logistic formula) and the cutoff value is 0.9872 (see FIG. 1A, predictors OHP and BMI, L* value, row 9). Two eligible girls take the test. Girl #1 has salivary OHP 0.057 ng/ml and BMI 19.8 kg/m2. The formula produces a logit value of 1.1186, larger than the threshold, so that we predict menarche within 60 days. The chance that this prediction proves correct is 40.2% (PPV), see FIG. 1A, column 8, predictors OHP and BMI. Girl #2 has salivary OHP 0.011 and BMI 20.1 kg/m2. The calculated logit is 0.7970, falling below the threshold and dictating a prediction of no menarche. The chance that 60 days will actually pass with no menarche for this girl is 93.4% (NPV), see FIG. 1A, column 10, predictors OHP and BMI.

Our intent in this application is to establish a proof of principle, that useful prediction formulas can be constructed from BMI and weekly or daily salivary hormone data for a population of girls (e.g. girls in Tanner Stage III-IV). In these pilot data the NPV is excellent, around 95%, and does not depend critically on the number or particular combination of hormones. The PPV is most consistent for 60-day prediction, falling around 50%.

We do not intend that these particular prediction formulas should be considered definitive, and we hold open the possibility that a more extensive data set may prove that optimal prediction can be made at some interval other than 60 days. The use of salivary hormones levels in the logistic equations is a distinct improvement on BMI alone, the latter having values barely better than random chance for ROC area and predictive value.

Examples III and IV provide two consumer diagnostic tools that are based on the predicted threshold H* values of the invention for predicting menarche onset within a specified time period.

### Example III

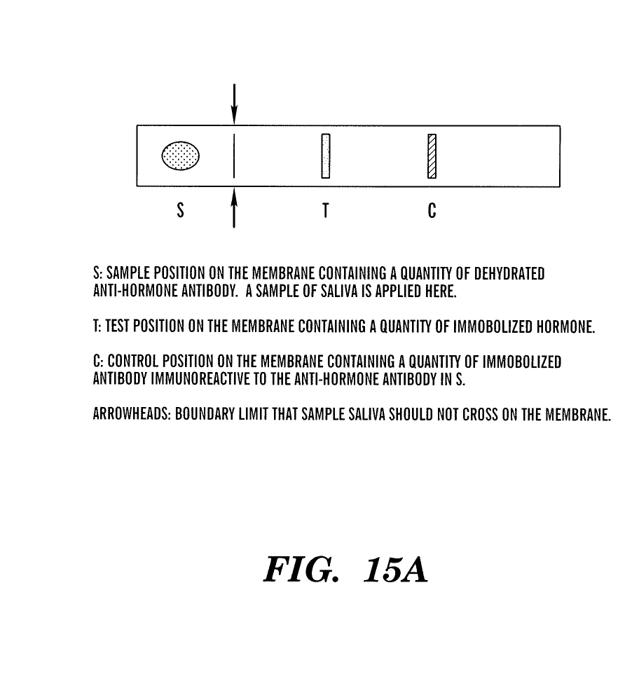

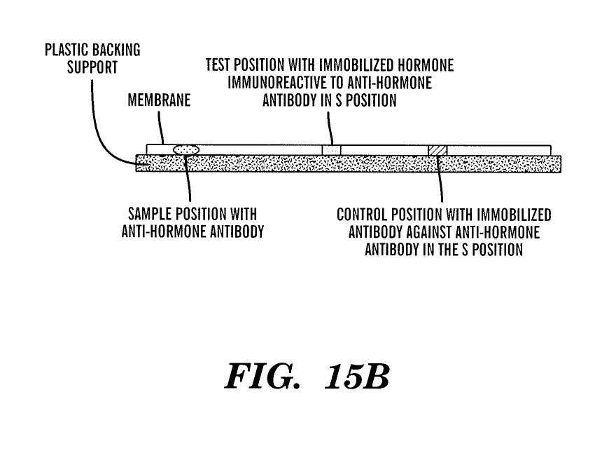

The levels of 17-β estradiol, testosterone, progesterone, and 17-α hydroxyprogesterone hormones in the saliva can be studied in a simple one-step analytical strategy illustrated in FIG. 15. In this one-step strategy, a single protein binding membrane strip is divided into three separate regions: a sample (S) position at one end of the membrane, a test (T) position located at the middle of the membrane, and a control (C) position found at the opposite end the membrane (FIG. 15). Located at S is a defined quantity of dehydrated anti-hormone antibody. The antibody can be conjugated to colloidal gold beads or latex beads for visualization purposes. At T, there is a defined quantity of hormone immobilized on the membrane. At C, there is another immobilized protein, an antibody immunoreactive to the anti-hormone antibody located at the S position (FIG. 15).

The defined quantity of dehydrated anti-hormone antibody at S position is such that there is just enough antibody to bind the ligand hormone from the saliva when the salivary hormone is at the threshold level. Therefore when the salivary hormone is at or above the threshold H* level, all the anti-hormone antibody at the S position will be bound to the hormone in the form of hormone-antibody conjugates; there will be no free anti-hormone antibody present.

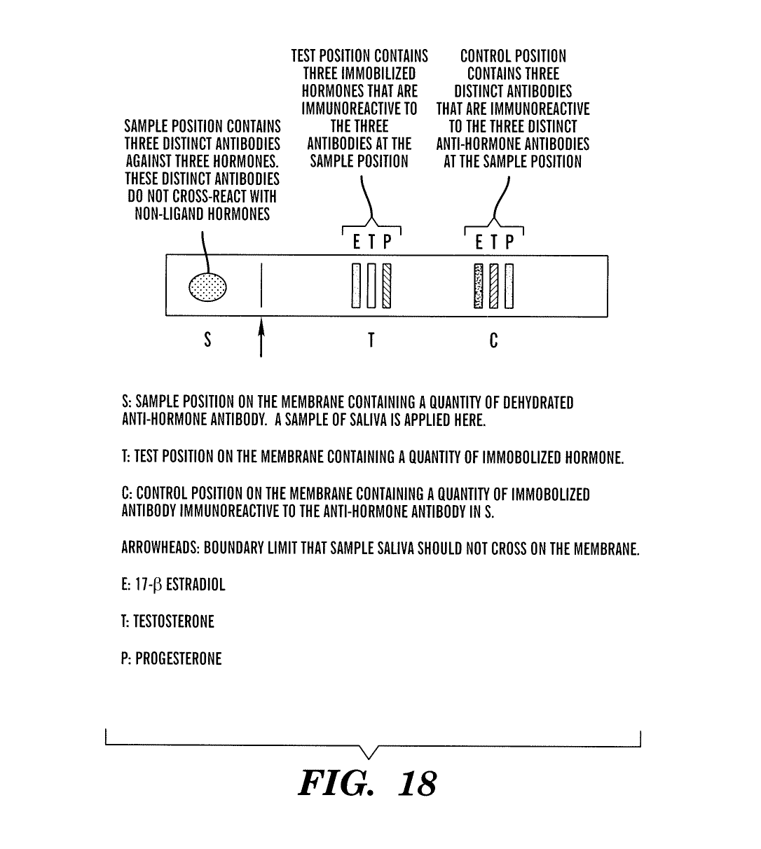

The choice of the anti-hormone antibody placed at the S position can be any antibody that is specially immunoreactive to any of the hormones of interest. The antibody can be monoclonal, polyclonal, or a mixture of both monoclonal and polyclonal antibodies. When only one hormone is studied, the S position will have only one antibody specially immunoreactive with just that one hormone of interest. On the other hand if three hormones are to be studied simultaneously, the S position will have three different types of anti-hormone antibodies, each type specially immunoreactive to one hormone and does not exhibit cross-reactivity with the other two non-ligand hormones (FIG. 18).

The S position is where a sample of saliva is applied. The arrowheads delineate the boundary limit that the sample saliva should not cross on the membrane when applying the saliva to the membrane.

The defined quantity of hormone immobilized on the membrane at the T position (FIG. 15) is a quantity that will bind and capture any free anti-hormone antibody from the S position that is not found in a hormone-antibody conjugate form. That quantity of immobilized hormone will serve to immobilize free anti-hormone antibody to the T position during testing. When only one hormone is studied, only one hormone, the same type as the one of interest will be immobilized at the T position. Likewise when three hormones are to be studied simultaneously, all three hormone types will be represented at the T position and at their respective quantities (FIG. 18).

The antibody at the C position can be a monoclonal, polyclonal, or a mixture of both monoclonal and polyclonal antibodies. When more than one salivary hormone is studied simultaneously, there will a corresponding number of anti-S-position antibodies and these antibodies should be specific for their respective ligand, the S position anti-hormone antibody, and should not cross-react with non-ligand (FIG. 18).

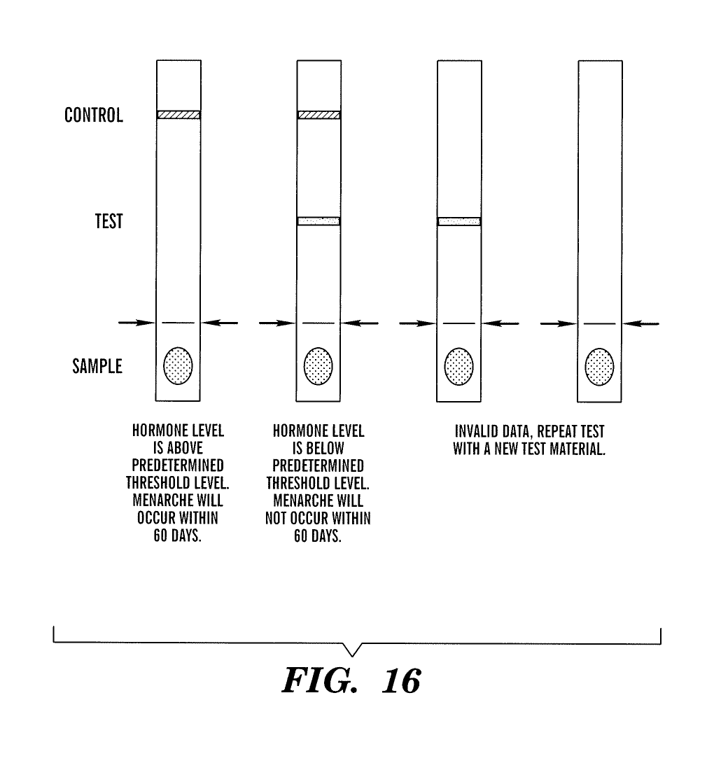

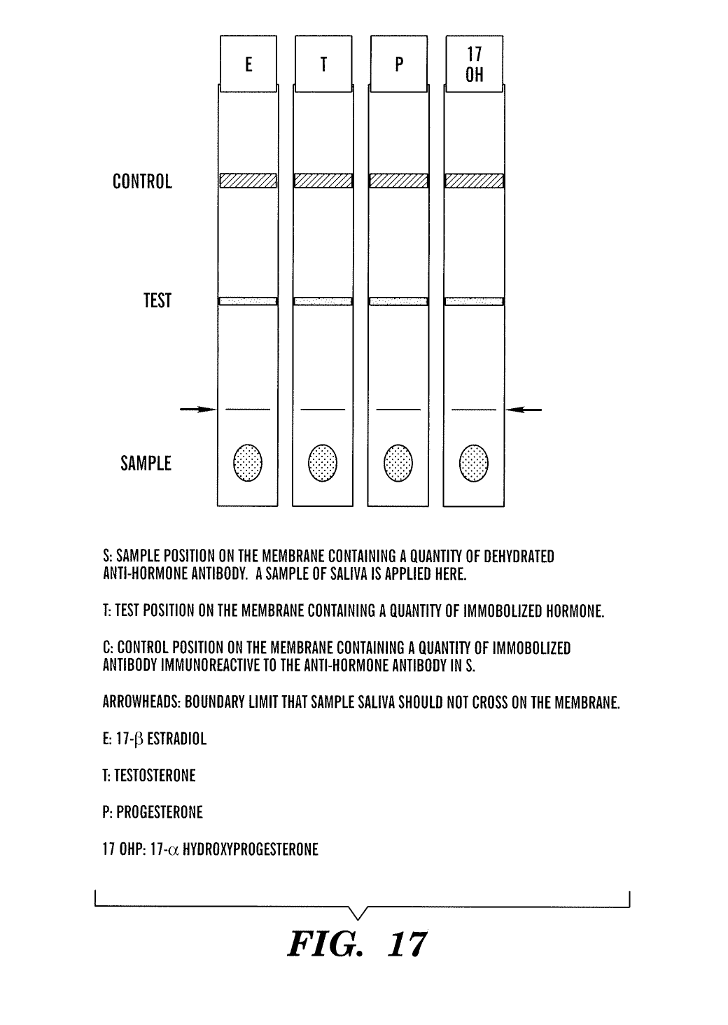

The application of a sample of saliva at the S position will re-hydrate the anti-hormone antibody and result in the antibody-hormone conjugate formation. By capillary action along the membrane, the fluid mixture of antibody and hormone will move toward the T position and subsequently to the C position. When the salivary hormone of interest is below the predetermined threshold level, the mixture of antibody and hormone will contain free anti-hormone antibody and antibody-hormone conjugates. Upon arrival at the T position, the free anti-hormone antibody will bind the immobilized hormone and be immobilized at the T position. The localized concentration of free anti-hormone antibody that is colloidal gold or latex bead labeled will become visible as a colored line at the T position (FIG. 16, middle). When the salivary hormone of interest is at or above the predetermined threshold H* level, the mixture of antibody and hormone will contain all antibody-hormone conjugates and no free anti-hormone antibody. At the T position, there will be no colloidal gold or latex bead labeled anti-hormone antibody, and the area remains clear (FIG. 16, left). At the C position the hormone-antibody conjugates will be bound and captured by the immobilized antibody immunoreactive against the anti-hormone antibody coming from the S position. This will in turn results in a concentration of a colloidal gold or latex bead labeled anti-hormone antibody at the C position and will become visible as colored line at the C position. The C position result serves as a test control that there is functional anti-hormone antibody in the test material and should always be present (FIG. 16, right).

The one-step analytical strategy of the invention may be designed in a form of a dipstick test strip (FIG. 15B). As a dipstick test strip, the strip is dipped into a sample of saliva at the S position end with saliva level not to exceed the boundary limit. The strip is then laid horizontally with the membrane surface facing up on a flat surface. A fixed amount of time is given for the antibody re-hydration, capillary action, and antibody binding reaction to take place. At the end of the fixed time, there should be visible bands at the C position and depending on the level of the hormone of interest, there may or may not be a visible band at the T position (FIG. 16). FIG. 3 shows a method of using four separate dipstick test strips to test for the four hormones of interest. Each dipstick test strip is labeled to indicate which hormone is being tested. FIG. 18 shows an alternate design where three hormones can be studied simultaneously on the same membrane and therefore on the same dipstick test strip. The positions of the expected results in the T and C positions for each hormone are indicated.

### Example IV

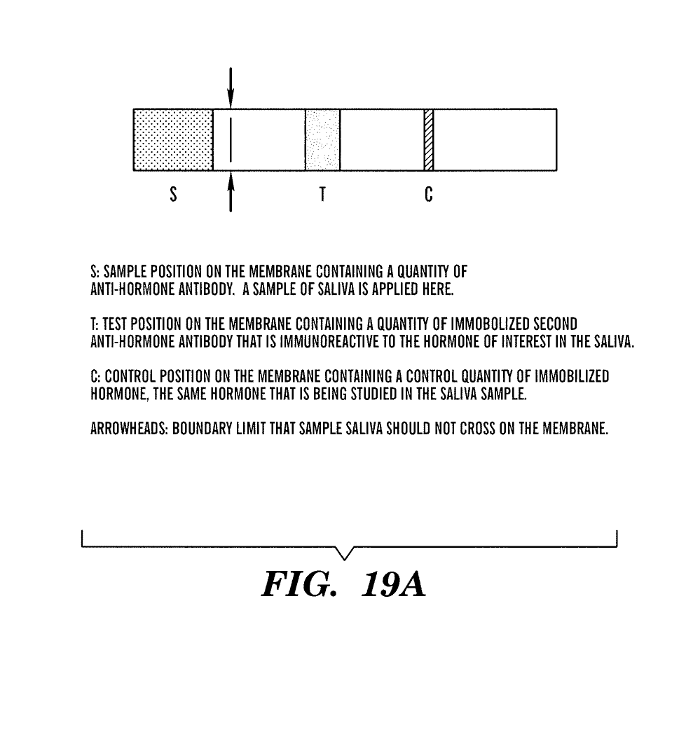

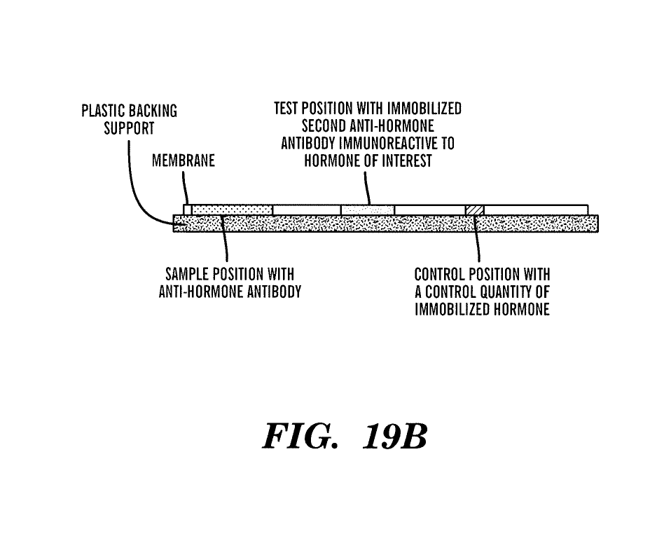

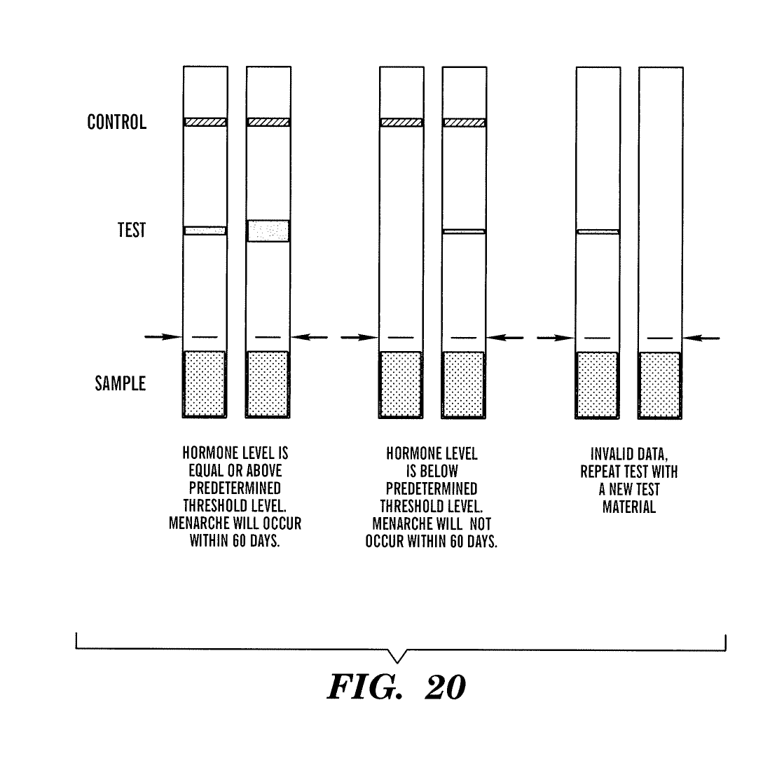

An alternate version of the simple one-step analytical strategy is illustrated in FIG. 5. There is the same single protein binding membrane strip that is divided into three separate regions: a sample (S), a test (T), and a control (C) positions, all three spatially arranged as shown in FIG. 15 and FIG. 19. The S position will contain an excess amount of dehydrate anti-hormone antibody that may be colloidal gold or latex bead labeled. The T position contains a second antibody that is also immunoreactive to the salivary hormone of interest. This second antibody is in excess and is immobilized on the membrane. This second anti-hormone antibody binds a part of the hormone that is different from the part of the hormone that is bound by the first anti-hormone antibody found at the S position. In this design, the second antibody at the T position will bind and capture both free unbound hormone and hormone-antibody conjugates, and concentrate them at the T position. The C position contains a defined quantity of hormone immobilized on the membrane. The defined quantity is the predetermined threshold level of the hormone under study. When the excess free anti-hormone from the S position arrives and bind the immobilized hormone, gradually becomes concentrated at the C position and will become visible as a colored line at the C position (FIG. 20).

An application of a sample of saliva at the S position will re-hydrate the excess amount of anti-hormone antibody there. A fluid mixture of free antibody and hormone-antibody conjugate is formed and will move along the membrane by capillary action towards the T position and then subsequently to the C position. At the T position, all hormones will be bound and immobilized, gradually become concentrated and visible as a colored line. With increasing amount of hormone bound and concentrated at the T position, the colored line expands and develop into a band. The greater the level of hormone of interest in the saliva, the wider the colored band at the T position (FIG. 20, left). When the excess free anti-hormone antibody from the S position arrives to the C position and bind to the immobilized hormone, anther color line become visible. Since there is a predetermined threshold amount of immobilized hormones at the C position, the thickness of the visible colored line at the C position defines the predetermined threshold amount of hormone. By comparing the thickness of the color band at the T and C positions on the same membrane, one can estimate whether the salivary hormone level is below or greater than the predetermined threshold level of the hormone. When the salivary hormone level is equal or greater than the threshold level, the color band at the T position will be equal or larger than the color band at the C position respectively (FIG. 20, left). When the salivary hormone level is below the threshold level, the color band at the T position will be smaller or even absent than the color band at the C position (FIG. 20, middle). The C position band also serves as a test control to confirm that there is functional anti-hormone antibody at the S position and that the functional anti-hormone antibody is derived from the S position (FIG. 20, right).

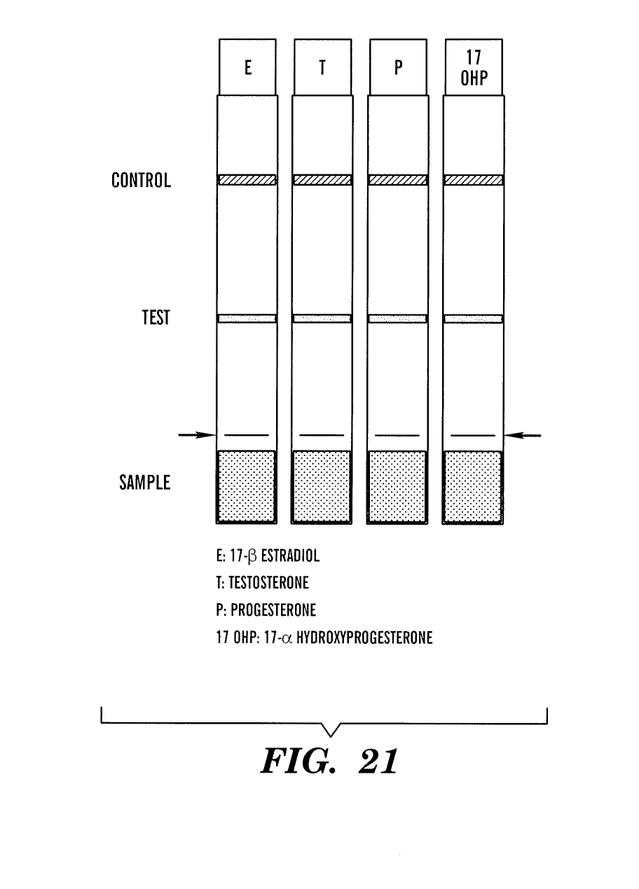

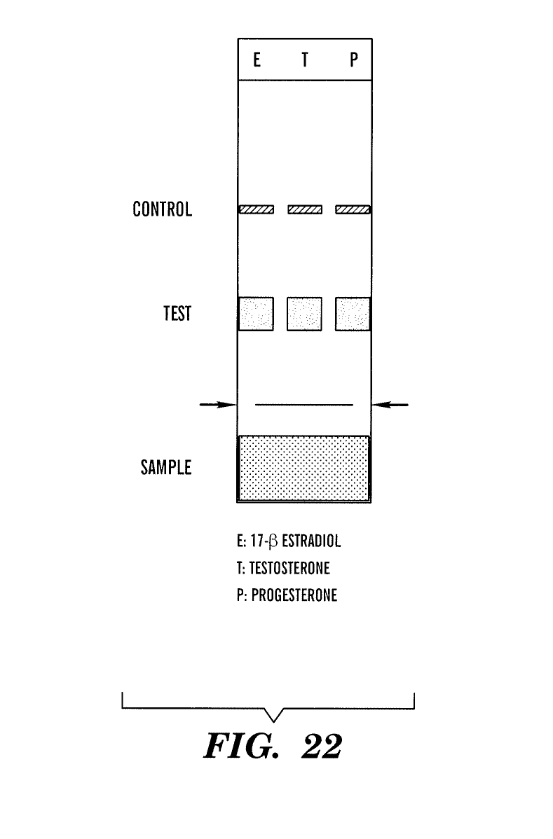

This alternate version of the simple one-step analytical strategy may be designed in the form of a dipstick test strip as shown in FIG. 21 where individual strip is used for each hormone and the strips are labeled. Another design is a single test strip where three hormones can be studied simultaneously on the same membrane. The positions of the expected results in the T and C positions for each hormone are indicated.

1.-59. (canceled)

60. A device for predicting that menarche is anticipated in a human female within a known time interval comprising a solid support and at least one antibody against at least one hormone in a body fluid, wherein the hormone is selected from the group consisting of testosterone, 17-beta estradiol, progesterone, 17-hydroxyprogesterone, and wherein said antibody is on the solid support.

61. The device of claim 60, wherein the hormones testosterone and 17-hydroxyprogesterone are selected.

62. The device of claim 60, wherein the hormones testosterone, 17-beta estradiol and progesterone are selected.

63. The device of claim 60, wherein the hormones testosterone, 17-beta estradiol and 17-hydroxyprogesterone are selected.

64. The device of claim 60, wherein the hormones progesterone, 17-beta estradiol and 17-hydroxyprogesterone are selected.

65. The device of claim 60, wherein the hormones testosterone, 17-beta estradiol, 17-hydroxyprogesterone, progesterone and are selected.

66. The device of claim 60, wherein said antibody is immobilized on the solid support.

67. The device of claim 60, wherein said antibody is of a defined quantity just enough to bind the hormone when the hormone is at a threshold level.

68. The device of claim 67, wherein the threshold level is selected from the group consisting of: a range between 0.01-0.05 nanograms per milliliter for testosterone, a range between 0.5-10.0 picograms per milliliter for 17-beta estradiol, a range between 0.01-0.05 nanograms per milliliter for progesterone, and a range between 0.01-0.10 nanograms per milliliter for 17-hydroxyprogesterone.

69. The device of claim 60, wherein said antibody is in excess of an amount just enough to bind the hormone when the hormone is at a threshold level.

70. The device of claim 69, wherein the threshold level is selected from the group consisting of: a range between 0.01-0.05 nanograms per milliliter for testosterone, a range between 0.5-10.0 picograms per milliliter for 17-beta estradiol, a range between 0.01-0.05 nanograms per milliliter for progesterone, and a range between 0.01-0.10 nanograms per milliliter for 17-hydroxyprogesterone.

71. The device of claim 60, wherein the antibody produces a detectable signal.

72. The device of claim 60, wherein said antibody is conjugated to colloidal gold beads or latex beads.

73. The device of claim 60, further comprising a second antibody against a hormone in a body fluid, wherein the hormone is selected from the group consisting of testosterone, 17-beta estradiol, progesterone, 17-hydroxyprogesterone, and wherein said second antibody is on the solid support.

74. The device of claim 60, wherein the body fluid is selected from a group consisting of saliva, tears, urine, sweat, blood plasma, or vaginal secretion.

75. The device of claim 60, wherein the device performs an immunoassay wherein an antibody-hormone complex is formed.

76. The device of claim 60, wherein the device is a dip stick or a test-strip.

77. The device of claim 60, wherein said time interval is between 1 to 60 days.

78. The device of claim 60, wherein said time interval is between 1 to 90 days.

79. The device of claim 60, wherein said time interval is between 1 to 30 days.

80. The device of claim 60, wherein said time interval is between 1 to 45 days.
